(19) United States
(12) Patent Application Publication
(10) Pub. No.: US 2025/0235429 A1
(43) Pub. Date: Jul. 24, 2025

(54) TREATMENT OF DISEASES VIA ADMINISTRATION OF BUNTANETAP AND AN ANTIDIABETIC AGENT

(71) Applicant: ANNOVIS BIO, INC., Malvern, PA (US)

(72) Inventor: Maria Maccecchini, West Chester, PA (US)

(73) Assignee: ANNOVIS BIO, INC., Malvern, PA (US)

(21) Appl. No.: 18/827,778

(22) Filed: Sep. 8, 2024

Related U.S. Application Data
(60) Provisional application No. 63/623,428, filed on Jan. 22, 2024.

(51) Int. Cl. A61K 31/407; A61K 9/00; A61K 38/26; A61P 25/28

(52) U.S. Cl. CPC A61K 31/407; A61K 9/0053; A61K 38/26; A61P 25/28

(57) ABSTRACT

The invention relates to methods and pharmaceutical compositions effective for treating, inhibiting, preventing, slowing, or delaying the onset of a neurodegenerative disease in mammals (e.g., humans) via the co-administration of an effective amount of a compound selected from the group consisting of Formula (I), Formula (II), Formula (III) or Formula (IV) or pharmaceutically acceptable salts thereof and an antidiabetic agent. In certain embodiments, the mammal is a type-1 diabetic, a type-2 diabetic, a type-3 diabetic, or has prediabetes.

This application claims the benefit of U.S. Provisional Application No. 63/623,428, filed on Jan. 22, 2024, hereby incorporated by reference.

### FIELD OF THE INVENTION

The present invention concerns methods of inhibiting, preventing, or treating pre-diabetes, diabetes, metabolic syndrome, obesity, inflammation, oxidative stress, neurodegenerative diseases, and dementia, in a human patient in need thereof via administration of (i) buntanetap, or a related compound, together with (ii) an antidiabetic agent, e.g., a GLP-1 agonist.

### BACKGROUND OF THE INVENTION

For unknown reasons, in a “sick” brain, the level of iron is high. The high level of iron in the “sick” brain induces iron regulatory protein 1 to release mRNAs coding for neurotoxic aggregating proteins causing upregulation of translation and synthesis. The upregulation of translation and synthesis of mRNAs coding for neurotoxic aggregating proteins leads to overproduction of these proteins in the “sick” brain, which leads, e.g., to impairment of axonal transport, inflammation, nerve cell death, and cognitive and motor function impairments.

Buntanetap is a translational inhibitor of multiple neurotoxic aggregating proteins.

Antidiabetic agents are primarily used to treat pre-diabetes, diabetes, and obesity.

Antidiabetic drugs are not currently approved by US FDA for treatment of neurodegenerative diseases.

### SUMMARY OF THE INVENTION

It is an object of the invention to provide a treatment for pre-diabetes, diabetes, metabolic syndrome, obesity, inflammation, oxidative stress, neurodegenerative diseases, and dementia.

It is a further object of the invention to provide a treatment for Alzheimer's disease and other forms of dementia (e.g., frontotemporal dementia), chronic traumatic encephalopathy, tauopathies, Parkinson's and alpha-synucleopathies, Prion's disease, Down Syndrome, Huntington's disease, multiple sclerosis, Amyloid Lateral Sclerosis, and other neurodegenerative diseases.

It is a further object of the invention to prevent, slow, or delay the development of neurodegenerative diseases.

In accordance with the above objects and others, the invention is directed in part to a method of treating a disease via administration of (i) buntanetap, or a compound that is similar to buntanetap as described herein, together with (ii) an antidiabetic agent to a human patient in need thereof, wherein an administered amount of at least one of these agents is at least 2 times lower than the amount of that agent that would be expected to provide any meaningful therapeutic effect if that agent were to be administered alone (i.e., as a monotherapy, without the other agent). Yet, the therapeutic effect provided by administration of (i) buntanetap, or the compound that is similar to buntanetap, together with (ii) the antidiabetic agent is greater than the sum of the therapeutic effects, if any, provided by administration of the same amount of buntanetap, or the compound that is similar to buntanetap, alone, and the therapeutic effect, if any, provided by administration of the same amount of the antidiabetic agent alone. In other words, in the methods of the invention, buntanetap boosts the therapeutic effect of the antidiabetic agent and/or the antidiabetic agent boost the therapeutic effect of buntanetap.

The invention is also directed in part to a method of treating a disease via administration of (i) buntanetap, or a compound that is similar to buntanetap, together with (ii) an antidiabetic agent to a human in need thereof, wherein the buntanetap and the antidiabetic agent are administered in amounts that are synergistic. In some of the embodiments, the therapeutic effect provided by the administration of the combination is at least 2 times greater than the expected additive effect of the combination.

The invention is also directed in part to a method of treating a disease via the administration of an amount of buntanetap, or the compound that is similar to buntanetap, together with an amount of an antidiabetic agent to a human patient in need thereof, wherein the amount of the antidiabetic agent is subtherapeutic when administered without buntanetap, or the compound that is similar to buntanetap, but is therapeutic when administered with the amount of buntanetap, or the compound that is similar to buntanetap.

The invention is also directed in part to a method of treating a disease via the administration of an amount of buntanetap, or the compound that is similar to buntanetap, together with an amount of an antidiabetic agent to a human patient in need thereof, wherein the amount of buntanetap, or the compound that is similar to buntanetap, is subtherapeutic when administered without the antidiabetic agent, but is therapeutic when administered with the amount of the antidiabetic agent.

The invention is further directed in part to a method of treating a disease via the administration of an amount of buntanetap, or the compound that is similar to buntanetap, together with an amount of an antidiabetic agent to a human patient in need thereof, wherein (i) the amount of buntanetap, or the compound that is similar to buntanetap, is subtherapeutic when administered without the antidiabetic agent, but is therapeutic when administered with the amount of the antidiabetic agent, and (ii) the amount of the antidiabetic agent is subtherapeutic when administered without buntanetap, or the compound that is similar to buntanetap, but is therapeutic when administered with the amount of buntanetap, or the compound that is similar to buntanetap.

In the methods of the invention, the administration of (i) buntanetap, or a compound that is similar to buntanetap, together with (ii) an antidiabetic agent may provide a therapeutic effect that is at least 2 to 30 times greater than the therapeutic provided by administration of the either agent alone. For example, in certain embodiments, an improvement provided by administration of buntanetap, or the compound that is similar to buntanetap, together with the antidiabetic agent may be 4 to 10 times greater than an improvement provided by administration of the antidiabetic agent alone. In certain embodiments, an improvement provided by administration of buntanetap, or the compound that is similar to buntanetap, together with the antidiabetic agent is 4 to 30 times greater than the improvement provided by administration of buntanetap alone. Thus, the improvement provided by the combination may, e.g., be about 3 times, 4 times, 5 times, 10 times, 15 times, 20 times, or 25 times greater than the improvement provided by administration of either agent alone.

The invention also encompasses a method of treating a disease via administration of (i) buntanetap, or a compound that is similar to buntanetap as described herein, together with (ii) an antidiabetic agent to a human in need thereof, wherein an administered amount of at least one of these agents is at least 2 times lower than the amount of that agent that would be expected or required to provide any meaningful therapeutic effect if that agent were to be administered alone.

The methods of the present invention may therefore allow for (i) administration of (a) a lower dose of buntanetap, or the compound that is similar to buntanetap, and/or (b) a lower dose of the antidiabetic agent, (ii) a reduction in incidence and/or severity of adverse effect(s), and/or (iii) an increased efficacy of (a) the buntanetap, or the compound that is similar to buntanetap, and/or (b) the antidiabetic agent.

For example, in some of the embodiments, the invention is directed to a method of treating a disease via administration of (i) buntanetap, or a compound that is similar to buntanetap as described herein, together with (ii) an antidiabetic agent to a human in need thereof, wherein buntanetap, or the compound that is similar to buntanetap, is administered in an amount that, per day, is less than 2 mg, less than 1 mg, less than 0.9 mg, or less than 0.8 mg. In some of these embodiments, the administered amount of buntanetap, or the compound that is similar to buntanetap, may e.g., be from about 0.01 mg to about 2 mg, from about 0.05 mg to about 1 mg or from about 0.05 mg to 0.9 mg, and could be administered orally, parenterally, or transdermally.

In additional embodiments, the invention is directed to a method of treating a disease via administration of (i) buntanetap, or a compound that is similar to buntanetap as described herein, together with (ii) an antidiabetic agent to a human in need thereof, wherein the antidiabetic agent is a GLP-1 agonist.

The invention is also directed in part to a method for treating a neurodegenerative disease comprising co-administering to a subject in need thereof: (i) buntanetap or a pharmaceutically acceptable salt thereof; and (ii) an antidiabetic agent; wherein buntanetap or the pharmaceutically acceptable salt thereof is administered in an amount from about 0.1 mg to about 60 mg, and the antidiabetic agent is administered in an amount from about 0.01 μg to about 3000 mg. In some of these embodiments, the antidiabetic agent is a GLP-1 agonist, including, e.g., dulaglutide, semaglutide, liraglutide, exenatide, lixisenatide, and pharmaceutically acceptable salts thereof. Buntanetap or the pharmaceutically acceptable salt thereof and the GLP-1 agonist may be independently administered orally, parenterally, intravenously, subcutaneously, sublingually, via suppository, nasally, topically, transdermally, or via an implant under the skin. For example, buntanetap or the pharmaceutically salt thereof may be administered orally, and the GLP-1 agonist may be administered subcutaneously. In one embodiment, buntanetap or the pharmaceutically salt thereof is administered once-a-day, and the GLP-1 agonist is be administered once-a-week. In some of these embodiments, the GLP-1 agonist is dulaglutide or a pharmaceutically acceptable salt thereof and is administered in an amount that, per week, is from 0.1 mg to 5 mg. In one embodiment, buntanetap or the pharmaceutically acceptable salt thereof is administered in an amount that, per day, is less than 0.9 mg. For example, buntanetap or the pharmaceutically acceptable salt thereof may be administered orally in an amount that, per day, is from about 0.01 mg to about 0.8 mg. In some of these embodiments, the amount of buntanetap or the pharmaceutically acceptable salt thereof that is administered would be subtherapeutic if administered alone without the GLP-1 agonist, and/or the amount of the GLP-1 agonist that is administered would be subtherapeutic is administered alone without buntanetap.

The invention is also directed in part to a method of treating a disease via administration of (i) buntanetap, or the compound that is similar to buntanetap, together with (ii) an antidiabetic agent to a human in need thereof, wherein the antidiabetic agent is semaglutide; buntanetap, or the compound that is similar to buntanetap, is administered in an amount that, per day, is less than 2 mg, less than 1 mg, less than 0.9 mg, or less than 0.8 mg; and semaglutide is administered in an amount that, per week, is from 0.1 mg to 3 mg. In some of these embodiments, buntanetap, or the compound that is similar to buntanetap, is administered orally once-a-day, and semaglutide is administered subcutaneously once-a-week.

The invention is also directed in part to a method of treating a disease via administration of (i) buntanetap, or a compound that is similar to buntanetap as described herein, together with (ii) an antidiabetic agent to a human in need thereof, wherein the antidiabetic agent is semaglutide, and the semaglutide dose per week is less than about 0.4 mg, less than about 0.3 mg or less than about 0.2 mg. In some of these embodiments, semaglutide is administered subcutaneously once-a-week in an amount that is less than about 0.4 mg, less than about 0.3 mg or less than about 0.2 mg, and buntanetap is administered orally once daily in amount from about 0.2 mg to about 60 mg, about 0.5 mg to about 50 mg, about 0.5 mg to about 30 mg, from about 0.5 mg to about 20 mg, from about 0.5 mg to about 10 mg or from about 0.5 mg to about 5 mg.

The invention is also directed in part to a method of treating a disease via administration of (i) buntanetap, or the compound that is similar to buntanetap, together with (ii) an antidiabetic agent to a human in need thereof, wherein the antidiabetic agent is semaglutide; buntanetap, or the compound that is similar to buntanetap, is administered orally once-a-day in an amount that, per day, is less than 2 mg, less than 1 mg, less than 0.9 mg, or less than 0.8 mg; and semaglutide is administered orally once-a-day in an amount from about 0.5 mg to about 50 mg.

The invention is also directed in part to a method of treating a disease via administration of (i) buntanetap, or the compound that is similar to buntanetap, together with (ii) an antidiabetic agent to a human in need thereof, wherein the antidiabetic agent is semaglutide; buntanetap, or the compound that is similar to buntanetap, is administered orally once-a-day in an amount that, per day, is from 0.1 mg to 80 mg; and semaglutide is administered orally once-a-day in an amount that is less than about 30 mg, 20 mg or 10 mg. In some of these embodiments, semaglutide is administered orally once-a-day at a dose of about 3 mg for at least 30 days; then, orally once-a-day at dose of about 7 mg for at least 30 days; and then orally once-a-day at a dose of about 14 mg.

The invention is also directed in part to a method of treating a disease via administration of (i) buntanetap, or the compound that is similar to buntanetap, together with (ii) an antidiabetic agent to a human in need thereof, wherein the antidiabetic agent is dulaglutide; buntanetap, or the compound that is similar to buntanetap, is administered in an amount that, per day, is less than 2 mg, less than 1 mg, less than 0.9 mg, or less than 0.8 mg; and dulaglutide is administered in an amount that, per week, is from 0.1 mg to 5 mg. In some of these embodiments, buntanetap, or the compound that is similar to buntanetap, is administered orally once-a-day, and dulaglutide is administered subcutaneously once-a-week.

The invention is also directed in part to a method of treating a disease via administration of (i) buntanetap, or a compound that is similar to buntanetap as described herein, together with (ii) an antidiabetic agent to a human in need thereof, wherein the antidiabetic agent is dulaglutide, and the dulaglutide dose per week is less than about 0.6 mg, less than about 0.5 mg or less than about 0.3 mg. In some of these embodiments, dulaglutide is administered subcutaneously once-a-week in an amount that is less than about 0.3 mg, less than about 0.2 mg or less than about 0.1 mg, and buntanetap is administered orally once daily in amount from about 0.2 mg to about 60 mg, about 0.5 mg to about 50 mg, about 0.5 mg to about 30 mg, from about 0.5 mg to about 20 mg, from about 0.5 mg to about 10 mg or from about 0.5 mg to about 5 mg.

The invention is also directed in part to a method of treating a disease via administration of (i) buntanetap, or the compound that is similar to buntanetap, together with (ii) an antidiabetic agent to a human in need thereof, wherein the antidiabetic agent is liraglutide; buntanetap, or the compound that is similar to buntanetap, is administered in an amount that, per day, is less than 2 mg, less than 1 mg, less than 0.9 mg, or less than 0.8 mg; and liraglutide is administered in an amount that, per day, is from 0.02 mg to 4 mg. In some of these embodiments, buntanetap, or the compound that is similar to buntanetap, is administered orally once-a-day, and liraglutide is administered subcutaneously once-a-day.

The invention is also directed in part to a method of treating a disease via administration of (i) buntanetap, or a compound that is similar to buntanetap as described herein, together with (ii) an antidiabetic agent to a human in need thereof, wherein the antidiabetic agent is liraglutide, and the liraglutide dose per week is less than about 0.4 mg, less than about 0.3 mg or less than about 0.2 mg. In some of these embodiments, liraglutide is administered subcutaneously once-a-week in an amount that is less than about 0.4 mg, less than about 0.3 mg or less than about 0.2 mg, and buntanetap is administered orally once daily in amount from about 0.2 mg to about 60 mg, about 0.5 mg to about 50 mg, about 0.5 mg to about 30 mg, from about 0.5 mg to about 20 mg, from about 0.5 mg to about 10 mg or from about 0.5 mg to about 5 mg.

The invention is also directed in part to a method of treating a disease via administration of (i) buntanetap, or the compound that is similar to buntanetap, together with (ii) an antidiabetic agent to a human in need thereof, wherein the antidiabetic agent is tirzepatide; buntanetap, or the compound that is similar to buntanetap, is administered in an amount that, per day, is less than 2 mg, less than 1 mg, less than 0.9 mg, or less than 0.8 mg; and tirzepatide is administered in an amount that, per week, is from 0.1 mg to 20 mg. In some of these embodiments, buntanetap, or the compound that is similar to buntanetap, is administered orally once-a-day, and tirzepatide is administered subcutaneously once-a-week.

The invention is also directed in part to a method of treating a disease via administration of (i) buntanetap, or a compound that is similar to buntanetap as described herein, together with (ii) an antidiabetic agent to a human in need thereof, wherein the antidiabetic agent is tirzepatide, and the tirzepatide dose per week is less than about 2 mg, less than about 1.5 mg or less than about 1 mg.

In certain embodiments, the antidiabetic agent is tirzepatide and is administered subcutaneously once-a-week in an amount that is less than about 2 mg, less than about 1 mg or less than about 0.5 mg, and buntanetap is administered orally once daily in amount from about 0.1 mg to about 50 mg, from about 0.2 mg to about 20 mg or from about 0.5 mg to about 5 mg.

The invention is also directed in part to a method of treating a disease via administration of (i) buntanetap, or the compound that is similar to buntanetap, together with (ii) an antidiabetic agent to a human in need thereof, wherein the antidiabetic agent is exenatide; buntanetap, or the compound that is similar to buntanetap, is administered in an amount that, per day, is less than 2 mg, less than 1 mg, less than 0.9 mg, or less than 0.8 mg; and exenatide is administered in an amount that, per day, is from 0.1 μg to 30 μg. In some of these embodiments, buntanetap, or the compound that is similar to buntanetap, is administered orally once-a-day, and exenatide is administered subcutaneously twice-a-day.

The invention is also directed in part to a method of treating a disease via administration of (i) buntanetap, or a compound that is similar to buntanetap as described herein, together with (ii) an antidiabetic agent to a human in need thereof, wherein the antidiabetic agent is exenatide, and the exenatide dose per day is less than about 8 μg, less than about 6 μg or less than about 4 μg.

In certain embodiments, the antidiabetic agent is exenatide and is administered subcutaneously twice-a-day in an amount that is less than about 4 μg, less than about 3 μg or less than about 1 μg, and buntanetap is administered orally once daily in amount from about 0.1 mg to about 50 mg, from about 0.2 mg to about 20 mg or from about 0.5 mg to about 5 mg.

In certain embodiments, the antidiabetic agent is exenatide and is administered subcutaneously once-a-week in an amount that is 2.5 mg or less, and buntanetap is administered orally once daily in amount from about 0.1 mg to about 50 mg, from about 0.2 mg to about 20 mg or from about 0.5 mg to about 5 mg. In some of these embodiments, exenatide and is administered subcutaneously once-a-week in an amount from about 1 to 2 mg, and buntanetap is administered orally once daily in amount from about 0.1 mg to about 30 mg, from about 0.2 mg to about 20 mg or from about 0.5 mg to about 5 mg.

The invention is also directed in part to a method of treating a disease via administration of (i) buntanetap, or the compound that is similar to buntanetap, together with (ii) an antidiabetic agent to a human in need thereof, wherein the antidiabetic agent is lixisenatide; buntanetap, or the compound that is similar to buntanetap, is administered in an amount that, per day, is less than 2 mg, less than 1 mg, less than 0.9 mg, or less than 0.8 mg; and lixisenatide is administered in an amount that, per day, is from 0.1 μg to 30 μg. In some of these embodiments, buntanetap, or the compound that is similar to buntanetap, is administered orally once-a-day, and lixisenatide is administered subcutaneously once-a-day.

The invention is also directed in part to a method of treating a disease via administration of (i) buntanetap, or a compound that is similar to buntanetap as described herein, together with (ii) an antidiabetic agent to a human in need thereof, wherein the antidiabetic agent is lixisenatide, and the lixisenatide dose per day is less than about 18 μg, less than about 10 μg or less than about 5 μg.

In certain embodiments, the antidiabetic agent is lixisenatide and is administered subcutaneously once-a-day in an amount that is less than about 8 μg, less than about 5 μg or less than about 3 μg, and buntanetap is administered orally once daily in amount from about 0.1 mg to about 50 mg, from about 0.2 mg to about 20 mg or from about 0.5 mg to about 5 mg.

The invention is also directed in part to a method of treating a disease via administration of (i) buntanetap, or the compound that is similar to buntanetap, together with (ii) an antidiabetic agent to a human in need thereof, wherein the antidiabetic agent is albiglutide; buntanetap, or the compound that is similar to buntanetap, is administered in an amount that, per day, is less than 2 mg, less than 1 mg, less than 0.9 mg, or less than 0.8 mg; and albiglutide is administered in an amount that, per week, is from 1 mg to 50 mg. In some of these embodiments, buntanetap, or the compound that is similar to buntanetap, is administered orally once-a-day, and albiglutide is administered subcutaneously once-a-week.

The invention is also directed in part to a method of treating a disease via administration of (i) buntanetap, or a compound that is similar to buntanetap as described herein, together with (ii) an antidiabetic agent to a human in need thereof, wherein the antidiabetic agent is albiglutide, albiglutide is administered subcutaneously, and the albiglutide dose per administration is less than about 25 mg, less than about 20 mg or less than about 10 mg.

The invention is also directed in part to a method of treating a disease via administration of (i) buntanetap, or a compound that is similar to buntanetap as described herein, together with (ii) an antidiabetic agent to a human in need thereof, wherein the antidiabetic agent is albiglutide, and the albiglutide is administered subcutaneously once-a-week at a dose that is less than about 25 mg, less than about 20 mg or less than about 15 mg, and buntanetap is administered orally once-a-day in amount from about 0.2 mg to about 60 mg, from about 0.5 mg to about 50 mg, from about 0.5 mg to about 30 mg, from about 0.5 mg to about 20 mg, from about 0.5 mg to about 10 mg or from about 0.5 mg to about 5 mg.

The invention is also directed in part to a method of treating a disease via the administration of (i) buntanetap, or the compound that is similar to buntanetap, together with (ii) an antidiabetic agent to a human patient in need thereof in amounts that provide a therapeutic effect that is greater than (i) a therapeutic effect provided by administration of buntanetap, or the compound that is similar to buntanetap, without the antidiabetic agent and/or (ii) a therapeutic effect that is greater than a therapeutic effect provided by administration of buntanetap, or the compound that is similar to buntanetap, without the antidiabetic agent.

Diseases that could be treated by the methods of the invention include, e.g., Alzheimer's disease, other forms of dementia (e.g., frontotemporal dementia), chronic traumatic encephalopathy, tauopathies, Parkinson's disease, alpha-synucleopathies, Prion's disease, Down Syndrome, Huntington's disease, multiple sclerosis, Amyloid Lateral Sclerosis, and other neurodegenerative diseases.

Depending on the disease that is treated, the “therapeutic effect” provided by the methods of the present invention may include, e.g., a reduction of and/or severity of a symptom of a neurodegenerative disease, an improvement in cognitive function (e.g., memory, cognition, etc.), an improvement in motor function, a reduction in fasting plasma glucose level; a reduction in plasma Hemoglobin A1C (HbA1c) level; a reduction in one or more of plasma inflammatory marker(s) (e.g., macrophage inhibitory cytokine-1 (MIC-1), C-reactive protein (CRP), interleukin-6 (IL6), soluble TNF receptor 2 (STNFR-2), erythrocyte sedimentation rate (ESR)) level(s); a reduction in AST (SGOT) plasma level; a reduction in ALT (SGPT) plasma level; a reduction in plasma Total Tau level; a decrease in the production of amyloid beta (A); a decrease in the production of tau; a decrease in the production of alpha-Synuclein (aSYN); a decrease in the production of TDP43; a reduction in weight; a reduction in blood pressure; and a combination of two or more of any of the foregoing.

The invention is directed in part to a method of treating a neurodegenerative disease via the administration of (i) buntanetap, or the compound that is similar to buntanetap, together with (ii) an antidiabetic agent to a human patient in need thereof, wherein the neurodegenerative disease is Alzheimer's disease, and the therapeutic effect is an improvement in cognition and mental status. The improvements in cognition and mental status may, e.g., be ascertained by a change in a score on a standardized test.

The invention is also directed in part to a method of treating a neurodegenerative disease via the administration of (i) buntanetap, or the compound that is similar to buntanetap, together with (ii) an antidiabetic agent to a human patient in need thereof, wherein the neurodegenerative disease is Parkinson's disease, and the therapeutic effect is an improvement in motor function. The improvement in motor function may be measured, e.g., by a change in a score on MDS-UPDRS or a part thereof.

The invention is further directed in part to a method of treating Parkinson's disease via the administration of (i) buntanetap, or the compound that is similar to buntanetap, together with (ii) an antidiabetic agent to a human patient in need, wherein the therapeutic effect are improvements e.g. in Postural Instability, Gait Difficulty, Tremors, or Cognition.

The invention is further directed in part to a method of treating diabetes via the administration of (i) buntanetap, or the compound that is similar to buntanetap, together with (ii) an antidiabetic agent to a human patient in need, wherein the therapeutic effect is a decrease in Hemoglobin A1c plasma level. In the methods of the invention, Hemoglobin A1c plasma level may, e.g., be ascertained after 3 months of administration.

The administration in accordance with the present invention preferably provides a therapeutic effect that is at least 4 times greater than (i) a therapeutic effect provided by administration of buntanetap, or the compound that is similar to buntanetap, alone without the antidiabetic agent and/or (ii) a therapeutic effect provided by administration of the antidiabetic agent alone without buntanetap, or the compound that is similar to buntanetap. In certain embodiments, the administration of the combination of (i) buntanetap, or the compound that is similar to buntanetap, and (ii) the antidiabetic agent in accordance with the present invention provides a therapeutic effect that is at least 5 times, 6 times, 7 times, 8, times, or 10 times greater than (i) a therapeutic effect, if any, provided by administration of buntanetap, or the compound that is similar to buntanetap, without the antidiabetic agent and/or (ii) a therapeutic effect, if any, provided by administration of the antidiabetic agent without buntanetap or the compound that is similar to buntanetap.

The invention is also directed in part to a method of treating a neurodegenerative disease via the administration of (i) buntanetap, or the compound that is similar to buntanetap, together with (ii) an antidiabetic agent to a human patient in need thereof in amounts that provide a therapeutic effect that is synergistic, wherein the neurodegenerative disease is Alzheimer's disease, and the therapeutic effect is an improvement in cognition and mental status. These improvements in mental status may, e.g., be measured by a change in an ADAS-Cog score after three months of the administration.

In the methods of the invention, buntanetap, or the compound that is similar to buntanetap, and the antidiabetic agent may be administered as free bases, pharmaceutically acceptable salts, metabolites, prodrugs, complexes, and mixtures thereof by the same or different route of administration and at the same or different times.

The invention is also directed in part to a method for treating, inhibiting, preventing, slowing, or delaying the onset of a neurodegenerative disease comprising co-administering to a subject in need thereof:
(1) an amount of a compound selected from the group consisting of Formula (I), Formula (II) and Formula (III):

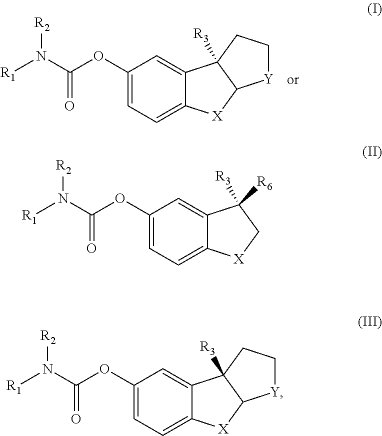

wherein,
in Formula (I) and Formula (II),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₅ alkenyl or C₂-C₈ alkynyl, aralkyl, or substituted or unsubstituted aryl; and
R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4;
   wherein,
in Formula (III),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X is NR₅, wherein R₅ is C₂₋₈ alkenyl, C₂₋₈ alkynyl, or aralkyl;
Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl and wherein the compound having the Formula (I), Formula (II) or Formula (III) is the substantially pure (−)-enantiomer, the substantially pure (+)-enantiomer, or a racemic mixture of the (−)-enantiomer and (+)-enantiomers or a pharmaceutically acceptable salt thereof; and (2) an amount of an antidiabetic agent. The compound and the antidiabetic agent may each be independently administered orally, parenterally, intravenously, subcutaneously, sublingually, via suppository, nasally, topically, transdermally, or via implant under the skin. In one embodiment, the compound is buntanetap or a pharmaceutically acceptable salt thereof. In one embodiment the compound is chronically administered to a subject with type 1 diabetes or type 2 diabetes, or prediabetes. In some of these embodiments, the compound is buntanetap or a pharmaceutically acceptable salt thereof, and the subject is not demonstrating symptoms of a neurological disorder or a neurodegenerative disease. Buntanetap may, e.g., be administered once-a-day orally in an amount from about 0.01 mg to about 2 mg or in an amount from about 0.01 mg to about 0.8 mg. In one embodiment, the administration provides a peak plasma circulating levels of buntanetap from about 1 ng/mL to about 40 ng/mL. The antidiabetic agent may be a GLP-1 agonist. In one embodiment, the GLP-1 agonist is dulaglutide or a pharmaceutically acceptable salt thereof. In one embodiment, the compound is buntanetap or a pharmaceutically acceptable salt thereof and is administered in an amount from about 0.1 mg to about 60 mg, and the antidiabetic agent is administered in an amount from about 0.01 μg to about 3000 mg. In one embodiment, the neurodegenerative disease is selected from a group consisting of Alzheimer's disease, a tauopathy, chronic traumatic encephalopathy, frontotemporal dementia, Parkinson's disease, an alpha-synucleopathy, Prion's disease, Down Syndrome, Huntington's disease, multiple sclerosis, and Amyloid Lateral Sclerosis.

The invention is also directed in part to a method for treating, inhibiting, preventing, slowing, or delaying the onset of a neurodegenerative disease comprising co-administering to the human:
(1) a therapeutically effective amount of a compound selected from the group consisting of Formula (I), Formula (II) and Formula (III):

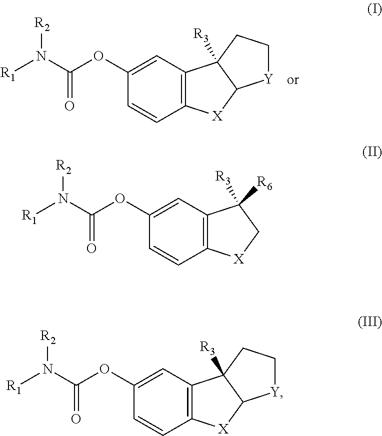

wherein,
in Formula (I) and Formula (II),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and
R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4;
   wherein,
in Formula (III),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X is NR₅, wherein R₅ is C₂₋₈ alkenyl, C₂₋₈ alkynyl, or aralkyl;
Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl and wherein the compound having the Formula (I), Formula (II) or Formula (III) is the substantially pure (−)-enantiomer, the substantially pure (+)-enantiomer, or a racemic mixture of the (−)-enantiomer and (+)-enantiomers or a pharmaceutically acceptable salt thereof; and
(2) a therapeutically effective amount of compound an antidiabetic agent; wherein the administration provides a synergistic therapeutic effect.

In certain embodiments, the compound selected from the group consisting of Formula (I), Formula (II) and Formula (III) is buntanetap or a pharmaceutically acceptable salt thereof, is administered orally once-a-day for at least three months at a dose of from about 0.01 mg to about 2 mg, from about 0.01 mg to about 1 mg or from about 0.01 mg to about 0.8 mg, and, after administration for at least three months, provides an increase in an ADAS-Cog score, as compared to an ADAS-Cog score at the start of the administration.

The invention is further directed in part to a method of treating a disease via the administration of buntanetap, or the compound that is similar to buntanetap, together with an antidiabetic agent to a human patient in need thereof in a weight ratio that provides a synergistic therapeutic effect, as compared to the administration of buntanetap without the antidiabetic agent and/or administration of the antidiabetic agent without buntanetap.

The invention is also directed in part to a method of treating a dementia via the administration of buntanetap, or the compound that are similar to buntanetap, together with an antidiabetic agent to a human patient in need thereof in amounts that provide a synergistic therapeutic effect, as compared to the administration of buntanetap without the antidiabetic agent and/or administration of the antidiabetic agent without buntanetap.

The invention is also directed in part to a method of treating Alzheimer's disease via the administration of buntanetap, or the compound that is similar to buntanetap, together with an antidiabetic agent to a human patient in need thereof in amounts that provide a synergistic therapeutic effect, as compared to the administration of buntanetap without the antidiabetic agent and/or administration of the antidiabetic agent without buntanetap.

The invention is also directed in part to a method of treating Parkinson's disease via the administration of buntanetap, or the compound that is similar to buntanetap, together with an antidiabetic agent to a human patient in need thereof in amounts that provide a synergistic therapeutic effect, as compared to the administration of buntanetap without the antidiabetic agent and/or administration of the antidiabetic agent without buntanetap.

The invention is also directed in part to a method of treating pre-diabetes via the administration of buntanetap, or the compound that is similar to buntanetap, together with an antidiabetic agent to a human patient in need thereof in amounts that provide a synergistic therapeutic effect, as compared to the administration of buntanetap without the antidiabetic agent and/or administration of the antidiabetic agent without buntanetap.

The invention is further directed in part to a method of treating diabetes via the administration of buntanetap, or the compound that is similar to buntanetap, together with an antidiabetic agent to a human patient in need thereof in amounts that provide a synergistic therapeutic effect, as compared to the administration of buntanetap without the antidiabetic agent and/or administration of the antidiabetic agent without buntanetap.

The invention is also directed in part to a method of treating metabolic syndrome via the administration of buntanetap, or the compound that is similar to buntanetap, together with an antidiabetic agent to a human patient in need thereof in amounts that provide a synergistic therapeutic effect, as compared to the administration of buntanetap without the antidiabetic agent and/or administration of the antidiabetic agent without buntanetap.

The invention is directed in part to a method of treating obesity via the administration of buntanetap, or the compound that is similar to buntanetap, together with an antidiabetic agent to a human patient in need thereof in amounts that provide a synergistic therapeutic effect, as compared to the administration of buntanetap without the antidiabetic agent and/or administration of the antidiabetic agent without buntanetap.

The invention is also directed in part to a method of treating inflammation via the administration of buntanetap, or the compound that is similar to buntanetap, together with an antidiabetic agent to a human patient in need thereof in amounts that provide a synergistic therapeutic effect, as compared to the administration of buntanetap without the antidiabetic agent and/or administration of the antidiabetic agent without buntanetap.

The invention is also directed in part to a method of treating oxidative stress via the administration of buntanetap, or the compound that is similar to buntanetap, together with an antidiabetic agent to a human patient in need thereof in amounts that provide a synergistic therapeutic effect, as compared to the administration of buntanetap without the antidiabetic agent and/or administration of the antidiabetic agent without buntanetap.

The invention is also directed in part to a method of treating a disease via the administration of buntanetap, or the compound that is similar to buntanetap, together with an antidiabetic agent selected from a group consisting of a glucagon-like peptide-1 receptor agonist (“GLP-1 RA” or “GLP-1 agonist”), a GLP-1/glucagon dual agonist, a dual glucose-dependent insulinotropic polypeptide (GIP) to a human patient in need thereof in amounts that provide a therapeutic effect that is greater than a therapeutic effect provided via administration of either agent alone.

The invention is also directed in part to a method of treating a disease via the administration of buntanetap, or the compound that is similar to buntanetap, together with an antidiabetic agent selected from a group consisting of a glucagon-like peptide-1 receptor agonist, a GLP-1/glucagon dual agonist, a dual glucose-dependent insulinotropic polypeptide and GLP-1 receptor agonist to a human patient in need thereof in amounts that provide a synergistic therapeutic effect, as compared to the administration of buntanetap without the antidiabetic agent and/or administration of the antidiabetic agent without buntanetap.

The invention is further directed in part to a method to inhibit, prevent or treat aggregation of a neurotoxic aggregating protein(s) via the administration of buntanetap, or the compound that is similar to buntanetap, together with an antidiabetic agent, and one or more pharmaceutically acceptable excipients in amounts that provide a synergistic therapeutic effect, as compared to the administration of buntanetap without the antidiabetic agent and/or administration of the antidiabetic agent without buntanetap. Examples of neurotoxic aggregating proteins suitable for treatment with the present invention include, e.g., APP (amyloid precursor protein), A3 (amyloid-β peptide, a fragment of APP), SOD (super oxide dismutase) proteins, Tau, alpha-synuclein (SNCA), transmissible spongiform encephalopathy (TSE) prions, TDP43, and huntingtin (HTT).

The invention is further directed in part to a method to inhibit, prevent or treat a neurodegenerative disease such as Alzheimer's disease via the administration of buntanetap, or the compound that is similar to buntanetap, together with an antidiabetic agent, and one or more pharmaceutically acceptable excipients.

The invention is further directed in part to a method to inhibit, prevent or treat a neurodegenerative disease such as Alzheimer's disease in a patient who has diabetes mellitus or prediabetes via the administration of buntanetap, or the compound that is similar to buntanetap, together with an (appropriate) antidiabetic agent, and one or more pharmaceutically acceptable excipients. By “appropriate” antidiabetic agent, it is meant that an insulin is administered as the antidiabetic agent in patients suffering from type 1 diabetes, or an agent for treating type 2 diabetes is administered to a type 2 diabetic patient or prediabetic patient.

The invention is further directed in part to a method to inhibit, prevent or treat a neurodegenerative disease such as Parkinson's disease via the administration of buntanetap, or the compound that is similar to buntanetap, together with an antidiabetic agent, and one or more pharmaceutically acceptable excipients.

The invention is further directed in part to a method to inhibit, prevent or treat a neurodegenerative disease such as multiple sclerosis via the administration of buntanetap, or the compound that is similar to buntanetap, together with an antidiabetic agent, and one or more pharmaceutically acceptable excipients.

The invention is further directed in part to a method to inhibit, prevent or treat a neurodegenerative disease such as a tauopathy via the administration of buntanetap, or the compound that is similar to buntanetap, together with an antidiabetic agent, and one or more pharmaceutically acceptable excipients.

The invention is further directed in part to a method to inhibit, prevent or treat a neurodegenerative disease such as an alpha-synucleopathy via the administration of buntanetap, or the compound that is similar to buntanetap, together with an antidiabetic agent, and one or more pharmaceutically acceptable excipients.

The invention is further directed in part to a method to inhibit, prevent or treat a neurodegenerative disease such as a dementia via the administration of buntanetap, or the compound that is similar to buntanetap, together with an antidiabetic agent, and one or more pharmaceutically acceptable excipients.

The invention is further directed in part to a method to inhibit, prevent or treat a neurodegenerative disease such as Prion's disease via the administration of buntanetap, or the compound that is similar to buntanetap, together with an antidiabetic agent, and one or more pharmaceutically acceptable excipients.

The invention is further directed in part to a method to inhibit, prevent or treat a neurodegenerative disease such as Down Syndrome via the administration of buntanetap, or the compound that is similar to buntanetap, together with an antidiabetic agent, and one or more pharmaceutically acceptable excipients.

The invention is further directed in part to a method to inhibit, prevent or treat a neurodegenerative disease such as Huntington's disease via the administration of buntanetap, or the compound that is similar to buntanetap, together with an antidiabetic agent, and one or more pharmaceutically acceptable excipients.

The invention is further directed in part to a method to inhibit, prevent or treat a neurodegenerative disease such as Amyloid Lateral Sclerosis via the administration of buntanetap, or the compound that is similar to buntanetap, together with an antidiabetic agent, and one or more pharmaceutically acceptable excipients.

The invention is further directed to a method to treat, inhibit, or slow the onset of neurological disorders or diseases such as Alzheimer's disease, tauopathies, chronic traumatic encephalopathy, frontotemporal dementia, Parkinson's and alpha-synucleopathies, Prion's disease, Down Syndrome, Huntington's disease, multiple sclerosis, Amyloid Lateral Sclerosis, and other dementias and neurodegenerative diseases in mammals (e.g., humans) comprising or consisting of administering to the human (i) a therapeutically effective amount of buntanetap, active metabolites of buntanetap, therapeutically effective analogues of buntanetap, compounds that are similar to buntanetap as described herein, pharmaceutically acceptable salts and complexes thereof, together with (ii) an antidiabetic agent, and one or more pharmaceutically acceptable excipients. In certain embodiments, buntanetap is administered orally on a once-a-day basis.

In certain embodiments, buntanetap is administered in an amount from about 0.01 to about 120 mg, and numbers in between these numbers.

In certain embodiments, an amount of buntanetap per day administered in the methods of the present invention may, e.g., be 0.01 mg, 0.02 mg, 0.05 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, or 5 mg. In some of these embodiments, buntanetap is administered orally in a dose from about, e.g., 0.01 mg to about 0.8 mg or from about 0.01 mg to about 0.6 mg.

In certain embodiments, the buntanetap dose is administered intravenously in an amount from about 0.01 to about 25 mg/day.

In certain embodiments, the buntanetap dose is administered intraperitoneally/intramuscularly (IP/IM) in a dose from about 0.03 mg/day to about 0.7 mg/day. In certain preferred embodiments, the buntanetap is administered together with an appropriate antidiabetic agent as described further herein in following paragraphs.

In certain embodiments of each of the methods described above, the oral pharmaceutical composition includes from about 0.01 mg to about 10 mg, from about 0.05 mg to about 5 mg, from about 0.1 mg to about 5 mg, from about 0.25 mg to about 5 mg, or from about 0.1 mg to about 1 mg buntanetap or a pharmaceutically acceptable salt thereof, the IP/IM pharmaceutical composition includes from about 0.03 to about 7 mg buntanetap or a pharmaceutically acceptable salt thereof, and the intravenous (IV) pharmaceutical formulation includes from about 0.01 to about 3 mg buntanetap or a pharmaceutically acceptable salt thereof.

In certain preferred embodiments of the methods described herein, peak plasma circulating levels of buntanetap in humans range, e.g., from about 0.01 ng/mL to about 40 ng/mL, in certain embodiments from about 0.2 ng/mL to about 2 ng/mL, and more preferably from about 0.3 ng/mL to about 12 ng/mL. In certain preferred embodiments, the peak plasma circulating level is reached within about 6 hours after administration of buntanetap to humans. In certain embodiments, the peak plasma circulating level is reached within about 3 hours after administration of buntanetap to the humans. In certain embodiments, the plasma circulating level of buntanetap is equal to or greater than about 0.01 ng/mL, 0.05 ng/mL, 0.1 ng/mL, 0.2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 mg/mL, 15 ng/mL, 16 ng/mL, 17 ng/mL, 18 ng/mL, 19 ng/mL, or 20 ng/mL for at least 9 hours, and preferably for at least 12 hours, after administration of buntanetap to humans. In certain embodiments, the half-life of buntanetap in cerebrospinal fluid after administering is about 12 hours, and the half-life of buntanetap in plasma after administering is about 5 hours.

With respect to each of the methods described above, buntanetap, a pharmaceutically acceptable salt thereof, and compounds that are similar to buntanetap as described herein may be administered, e.g., orally, parenterally, sublingually, via suppository, nasally, topically, transdermally, or via implant under the skin.

In certain preferred embodiments, the antidiabetic agent is administered in an amount from about 0.01 μg to about 3000 mg, and any numbers in between these numbers. The antidiabetic agent may be administered orally, parenterally, sublingually, via suppository, nasally, topically, transdermally, or via implant under the skin.

The invention is further directed in part to a method for preventing, treating, inhibiting, or slowing a neurodegenerative disease such as Alzheimer's disease in a healthy human who is at risk of developing such a neurodegenerative disease comprising administering to the human an antidiabetic agent together with a compound selected from the group consisting of Formula (I), Formula (II), Formula (III) and Formula (IV):

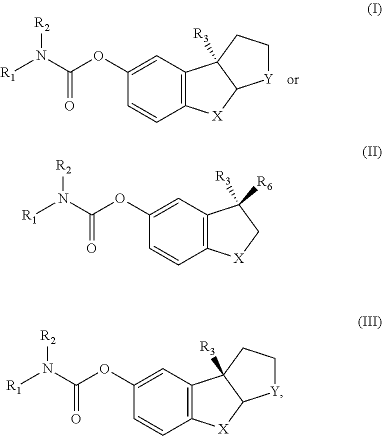

In Formula (I) and Formula (II), R₁ and R₂ are, independently, hydrogen, branched or straight chain C₁-C₈ alkyl, substituted or unsubstituted aryl, heteroaryl, or aralkyl; R₃ is branched or straight chain C₁-C₄ alkyl or heteroalkyl or C₄-C₈ alkyl or heteroalkyl, or substituted or unsubstituted aryl; X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and R₆ is hydrogen, C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4. In certain embodiments, the invention is directed to preventing or slowing the ability of melanoma cells to produce their own supply of Abeta with the treatment (administration of buntanetap) as set forth in this paragraph. In certain embodiments, the method includes the use of a second therapeutic active agent to treat, prevent, delay, or slow the onset of neurodegenerative disease such as Alzheimer's disease.

In certain embodiments, the compound selected from the group consisting of Formula (I), Formula (II), Formula (III) and Formula (IV) is devoid of any significant acetylcholinesterase (ACHE) inhibitory activity and devoid of any butyrylcholinesterase (BCHE) activity.

In certain embodiments, the compound of Formula (I) and Formula (II) is the a (+)-enantiomer. In other embodiments, the compound of Formula (I) and Formula (II) is a (−)-enantiomer.

In certain embodiments, the compound of Formula (I) is buntanetap or its active metabolites.

In preferred embodiments, the compound is buntanetap of Formula IV as follows:

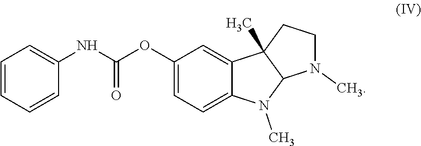

In Formula (III), R₁ and R₂ are, independently, hydrogen, branched or straight chain C₁-C₈ alkyl, substituted or unsubstituted aryl, heteroaryl, or aralkyl; R₃ is branched or straight chain C₁-C₄ alkyl or heteroalkyl or C₄-C₈ alkyl or heteroalkyl, or substituted or unsubstituted aryl; X is NR₅, wherein R₅ is C₂₋₈ alkenyl, C₂₋₈ alkynyl, or aralkyl, and Y is selected from C(H)R₄ or NR₅, wherein R₄ and R₅ are, independently, hydrogen, branched or straight chain C₁₋₈alkyl or heteroalkyl, alkenyl, or C₂-C₈ alkynyl, aralkyl.

The invention is further directed in part to a method for treating, preventing, delaying, or slowing a neurodegenerative disease such as Alzheimer's disease comprising administering to the human a compound selected from the group consisting of Formula (I), Formula (II), Formula (III) or Formula (IV) together with the antidiabetic agent as described herein.

The compounds are administered either separately or together via a route selected from the group consisting of orally, parenterally, sublingually, via suppository, nasally, topically, transdermally, intravenously, subcutaneously, intraperitoneally and via implant under the skin. In certain embodiments, the compound is administered chronically to treat, prevent, delay, or slow a neurodegenerative disease such as Alzheimer's disease in a mammal, e.g., a human. In certain embodiments, the neurological disorders or diseases encompass but are not limited to Alzheimer's disease, tauopathies, chronic traumatic encephalopathy, frontotemporal dementia, Parkinson's and alpha-synucleopathies, Prion's disease, Down Syndrome, Huntington's disease, multiple sclerosis, Amyloid Lateral Sclerosis, and other dementias and neurodegenerative diseases in mammals (e.g., humans).

In certain preferred embodiments, the co-administered compounds are buntanetap and the buntanetap is administered in an amount as set forth in this disclosure, along with an antidiabetic agent used to treat type-1 diabetes, type-2 diabetes, type-3 diabetes, or prediabetes, depending upon the patient's condition.

In certain embodiments, the compounds are co-administered to a mammal (e.g., human) who is experiencing type 1 diabetes, type 2 diabetes, type 3 diabetes, or prediabetes. In certain embodiments, the mammal (e.g., human subject or human patient) is not demonstrating symptoms of a neurological disorder or a neurodegenerative disease.

In certain embodiments, buntanetap or a pharmaceutically acceptable salt thereof may be administered (i) orally in an amount from about 0.01 mg to about 12 mg on a once-a-day basis; (ii) intravenously in an amount from about 0.01 mg to about 2.5 mg/day; or (ii) intraperitoneally/intramuscularly (IP/IM) in a dose from about 0.03 to about 7 mg/day.

The peak plasma circulating levels of buntanetap in humans provided by administration in accordance with the methods of the present invention may, e.g., range from about 0.1 ng/mL to about 40 ng/mL.

In certain embodiments, the compound of Formulas (I), (II), and (III), and the antidiabetic agent(s) are administered separately but such that they provide overlapping therapeutic effects.

In certain embodiments, the antidiabetic agent (compound) is an insulin and the patient has type-1 diabetes. In other embodiments, the antidiabetic agent (compound) is selected from the group consisting of a glucagon-like peptide-1 receptor agonist, a GLP-1/glucagon dual agonist, a dual glucose-dependent insulinotropic polypeptide, a sodium-glucose cotransporter 2 (SGLT2) inhibitor, a dipeptidyl peptidase-4 (DPP-4) inhibitor, a biguanide, a sulfonylurea, sodium glucose cotransporter 2 inhibitor, alpha-glucosidase inhibitor, dopamine-2 agonist, a meglitinide, a thiazolidinedione, an amylinomimetic, a type-3 antidiabetic agent, an androstenetriol or a similar compound, and PPAR activators, and combinations of any of the foregoing, and the mammal (e.g., human subject or human patient) is a type-2 diabetic, type-3 diabetic, or has prediabetes.

In certain embodiments, the antidiabetic agent is a GLP-1 agonist.

In some of the embodiments, the GLP-1 agonist may be administered subcutaneously once-a-day, twice-a-day or once-a-week in an amount from about 0.01 μg to about 10 mg, and buntanetap is administered orally once daily in amount from about 0.01 mg to about 120 mg, about 0.05 mg to about 80 mg, about 0.1 mg to about 60 mg, about 0.1 mg to about 30 mg, from about 0.1 mg to about 30 mg, from about 0.1 mg to about 20 mg, from about 0.1 mg to about 10 mg, from about 0.1 mg to about 5 mg, from about 0.1 mg to about 2 mg, from about 0.1 mg to about 1 mg, from about 0.1 mg to about 0.9 mg, from about 0.1 mg to about 0.8 mg, or from about 0.1 mg to about 0.7 mg.

The invention is also directed in part to a pharmaceutical composition, comprising: an amount of a compound selected from the group consisting of Formula (I), Formula (II) and Formula (III):

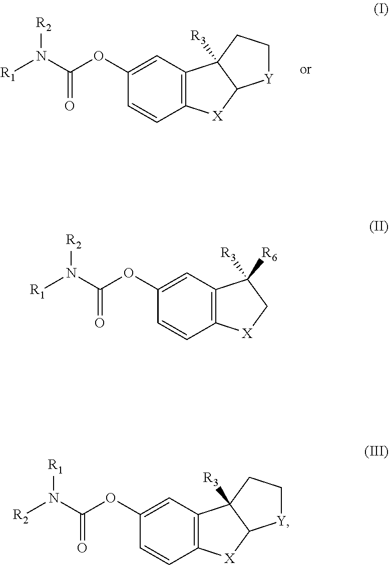

wherein,
in Formula (I) and Formula (II),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and
R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4;
   wherein,
in Formula (III),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X is NR₅, wherein R₅ is C₂₋₈ alkenyl, C₂₋₈ alkynyl, or aralkyl;
Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl and wherein the compound having the Formula (I), Formula (II) or Formula (III) is the substantially pure (−)-enantiomer, the substantially pure (+)-enantiomer, or a racemic mixture of the (−)-enantiomer and (+)-enantiomers or a pharmaceutically acceptable salt thereof; and
an amount of an antidiabetic agent. In some of these embodiments, the compound is buntanetap or a pharmaceutically acceptable salt thereof, and the antidiabetic agent is a glucagon-like peptide-1 receptor agonist.

In certain embodiments, the invention is further directed to a pharmaceutical composition, comprising a therapeutically effective amount of a compound selected from the group consisting of Formula (I), Formula (II) and Formula (III):

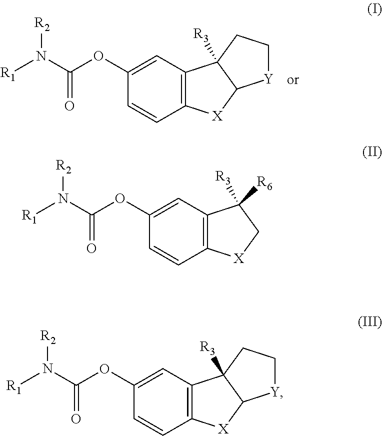

wherein,

### in Formula (I) and Formula (II),

R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₅ alkyl or heteroalkyl, or substituted or unsubstituted aryl;
X and Y are, independently, O, S, alkyl, hydrocarbon moiety, C(H)R₄, or NR₅, wherein R₄ and R₅ are, independently, hydrogen, oxygen, branched or straight chain C₁-C₅ alkyl, C₂-C₅ alkenyl or C₂-C₅ alkynyl, aralkyl, or substituted or unsubstituted aryl; and
R₆ is hydrogen; C₁-C₅ alkyl, C₁-C₅ alkenyl, C₂-C₅ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4;
   wherein,

### in Formula (III),

R₁ and R₂ are, independently, hydrogen, branched or straight chain C₁-C₅ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X is NR₅, wherein R₅ is C₂₋₈ alkenyl, C₂₋₈ alkynyl, or aralkyl;
Y is selected from C(H)R₄ or NR₅, wherein R₄ and R₅ are, independently, hydrogen, branched or straight chain C₁₋ₛ alkyl or heteroalkyl, alkenyl, or C₂-C₈ alkynyl, aralkyl and wherein the compound having the Formula (I), Formula (II) or Formula (III) is the substantially pure (−)-enantiomer, the substantially pure (+)-enantiomer, or a racemic mixture of the (−)-enantiomer and (+)-enantiomers or a pharmaceutically acceptable salt thereof;
an effective amount of an antidiabetic agent;
and at least one pharmaceutically acceptable excipient.

In certain preferred embodiments, the compound of Formula (III) is the substantially pure (−)-enantiomer. In certain embodiments, the compound is buntanetap of Formula (IV),

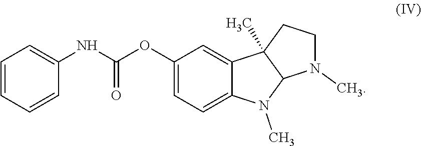 [SMILES: CN1CC[C@]2(C)c3cc(OC(=O)Nc4ccccc4)ccc3N(C)C12]

wherein the compound of Formula (IV) is the substantially pure (+)-enantiomer, or a pharmaceutically acceptable salt thereof. In certain embodiments, the buntanetap or a pharmaceutically acceptable salt thereof is in an amount from about 0.01 mg to about 50 mg, and more preferably from about 0.01 mg to about 30 mg, and more preferably in the about from about 0.01 mg to about 2 mg or from about 0.01 mg to about 0.8 mg. In certain embodiments, the antidiabetic agent is a therapeutically effective amount of a glucagon-like peptide-1 receptor agonist, a GLP-1/glucagon dual agonist, a dual glucose-dependent insulinotropic polypeptide, a sodium-glucose cotransporter 2 inhibitor, a dipeptidyl peptidase-4 inhibitor, a biguanide, a sulfonylurea, sodium glucose cotransporter 2 inhibitor, alpha-glucosidase inhibitor, dopamine-2 agonist, a meglitinide, a thiazolidinedione, an amylinomimetic, a type-3 antidiabetic agent, and combinations of any of the foregoing. In certain embodiments, the antidiabetic agent is included in a subtherapeutic amount. In certain embodiments, the pharmaceutical composition is an oral dosage form. In certain embodiments, the amount of the compound of Formula (I), (II), and (III), and the antidiabetic agent is effective in treating, inhibiting, preventing, slowing, or delaying the onset of a neurodegenerative disease (e.g., Alzheimer's disease). In certain embodiments, the neurological disorders or diseases encompass but are not limited to Alzheimer's disease, Tauopathies, chronic traumatic encephalopathy, frontotemporal dementia, Parkinson's and alpha-synucleopathies, Prion's disease, Down Syndrome, Huntington's disease, multiple sclerosis, Amyloid Lateral Sclerosis, and other dementias and neurodegenerative diseases in mammals (e.g., humans).

The invention is also directed in part to an oral dosage form comprising: (i) buntanetap or a pharmaceutically acceptable salt thereof, (ii) an antidiabetic agent and (iii) one or more pharmaceutically acceptable ingredient(s), wherein the oral dosage form contains from about 0.1 mg to about 60 mg of buntanetap or the pharmaceutically acceptable salt thereof and from about 0.1 mg to about 50 mg of the antidiabetic agent. In some of these embodiments, the antidiabetic agent is a GLP-1 agonist. In one embodiment, the GLP-1 agonist is semaglutide or a pharmaceutically acceptable salt thereof. In one embodiment, the oral dosage form contains from about 0.5 mg to about 50 mg of buntanetap or the pharmaceutically acceptable salt thereof.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in biochemistry, analytical chemistry and organic chemistry are those well-known and commonly employed in the art. Standard techniques or modifications thereof are used for chemical syntheses and chemical analyses.

The articles “a” and “an” are used herein to refer to one or to more than one (i.e. to at least one) of the grammatical objects of the article. By way of example, “an element” means one element or more than one element.

The term “about” will be understood by persons of ordinary skill in the art and will vary to some extent on the context in which it is used. In the context of the present application, the term “about” means a value within 20% (±20%) of the value recited immediately after the term “about,” including the value equal to the upper limit (i.e., +20%) and the value equal to the lower limit (i.e.,−20%) of this range. For example, the phrase “about 100” encompasses any numeric value that is between 80 and 120, including 80 and 120.

The term “an antidiabetic agent” means a drug that could be used to treat prediabetes or diabetes. In all of the embodiments of the invention, “an antidiabetic agent” includes GLP-1 agonists.

As used herein, the terms “Buntanetap and “Posiphen” are used interchangeably to refer to (3aR)-1,3a,8-trimethyl-1,2,3,3a,8,8a-hexahydropyrrolo[2,3-b]indol-5-yl phenylcarbamate or a salt thereof.

As used herein, the term “APP” refers to amyloid precursor protein.

As used herein, the term “Ap” refers to Abeta or amyloid beta or amyloid-β peptide.

For purposes of the present invention, these terms are considered to be synonymous.

For purposes of the present invention, a “Buntanetap-type” drug or “a compound that is similar to buntanetap” encompass Formula (I), Formula (II), Formula (III) or Formula (IV). In all embodiments described herein, “a compound that is similar to buntanetap” encompasses or consists of a pharmaceutically acceptable salt of buntanetap.

As used herein, the term “HTT” refers to huntingtin or the Huntington protein.

As used herein, “TDP43” refers to the TAR-DNA binding protein TDP43.

As used herein, “C9orf72” refers to the C9orf72 protein found in many regions of the brain.

As used herein, the term “neurotoxic aggregating protein” refers to a protein or family of proteins that has neurotoxic effect upon accumulating in a tissue of the brain. Non-limiting examples of neurotoxic aggregating proteins are APP, Aβ, SOD1, SNCA, NAC, TSE amyloid plaque, HTT, Tau, TDP43 and C9orf72.

As used herein, the terms “protein”, “peptide” and “polypeptide” are used interchangeably and refer to a compound comprised of amino acid residues covalently linked by peptide bonds.

By the term “specifically binds,” as used herein, is meant a molecule, such as an antibody or a small molecule, which recognizes and binds to another molecule or feature, but does not substantially recognize or bind other molecules or features in a sample.

The term “inhibit” as used herein means to reduce a molecule, a reaction, an interaction, a gene, an mRNA, and/or a protein's expression, stability, function, or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function, and activity, e.g., antagonists.

In the context of the invention, the “improvement in mental function” may be ascertained by a change in (i) a score on Alzheimer's Disease Assessment Scale-Cognitive Subscale (e.g., “ADAS-Cog 11”, “ADAS-Cog 12”, “ADAS-Cog 13”, “ASAS-Cog 14”) and/or (ii) a score on a Mini-Mental State Examination (MMSE) and/or (iii) a score on a Montreal Cognitive Assessment (MoCA) test and/or (iv) Wechsler Intelligence Test and/or (v) another scale (e.g., a computer scale) that assesses mental function, after 3 months of treatment, as compared to a score at the start of treatment, which is used as a baseline.

“Effective amount” or “therapeutically effective amount” are used interchangeably herein, and refer to an amount of a compound, formulation, material, or composition, as described herein effective to achieve a particular biological result. Such results may include, but are not limited to, the treatment of a disease or condition as determined by any means suitable in the art.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary, and topical administration.

As used herein, the term “co-administering” refers to a compound of Formula (I), (II), and/or (III) which is administered to a mammal (e.g., a human subject or human patient) together with an appropriate antidiabetic agent so that the two classes of agents provide an overlapping effect. While in certain cases it may be possible to administer the two classes of agents in a single dosage form, it is contemplated that these agents may be separately administered either via the same route of administration or different routes of administration to achieve overlapping effects, taking into account their differing physical/chemical properties (including but not limited to solubility, bioavailability, half-life, metabolism, and clearance/elimination from the body, etc.).

“Pharmaceutically acceptable” refers to a material(s) which are compatible with the activity of the compound useful within the invention and which are physiologically acceptable to the patient (e.g., human) from a pharmacological/toxicological point of view and to the manufacturing pharmaceutical chemist from a physical/chemical point of view regarding composition, formulation, stability, patient acceptance and bioavailability.

“Pharmaceutical acceptable carrier” refers to a pharmaceutically acceptable material, composition, or carrier, such as a liquid or solid filler, stabilizer, dispersing agent, suspending agent, diluent, excipient, thickening agent, solvent, or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “salt” embraces addition salts of free acids or free bases that are compounds useful within the invention. Suitable acid addition salts may be prepared from an inorganic acid or from an organic acid. Examples of inorganic acids include hydrochloric, hydrobromic, hydriodic, nitric, carbonic, sulfuric, phosphoric acids, perchloric and tetrafluoroboronic acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, O-hydroxybutyric, salicylic, galactaric and galacturonic acid. Suitable base addition salts of compounds useful within the invention include, for example, metallic salts including alkali metal, alkaline earth metal and transition metal salts such as, for example, lithium, calcium, magnesium, potassium, sodium, and zinc salts. Acceptable base addition salts also include organic salts made from basic amines such as, for example, N,N′-dibenzylethylenediamine, chloroprocaine, choline, diethanolamine, ethylenediamine, meglumine (N-methyl-glucamine) and procaine. All of these salts may be prepared by conventional means from the corresponding free base compound by reacting, for example, the appropriate acid or base with the corresponding free base.

An “individual”, “patient”, or “subject”, as that term is used herein, includes a member of any animal species including, but are not limited to, birds, humans and other primates, and other mammals including commercially relevant mammals such as cattle, pigs, horses, sheep, cats, and dogs. Preferably, the subject is a human.

The term neurologic disease or disorder is meant to include Alzheimer's disease, Tauopathies, Parkinson's and alpha-synucleopathies, Prion's disease, Down Syndrome, Huntington's disease, multiple sclerosis, Amyloid Lateral Sclerosis and other dementias and neurodegenerative diseases.

The term “synergy” as used herein means that the amount (i.e., dosage) of each agent alone has no effect and is sub-therapeutic as monotherapy but when administered as a combination is therapeutic.

The term “treat” or “treating” as used herein means reducing the frequency with which symptoms are experienced by a subject or administering the combination of agents or compounds to reduce the frequency and/or severity with which symptoms are experienced. As used herein, “alleviate” is used interchangeably with the term “treat.” Treating a disease, disorder or condition may or may not include complete eradication or elimination of the symptom. Thus, the term “treating” encompasses preventing and slowing a condition described herein.

The term “therapeutic” as used herein means a treatment and/or prophylaxis of a condition or disease state as described herein.

The term “alkyl” as used herein refers to a branched or unbranched saturated hydrocarbon group of 1 to 4, 1 to 8, or 1 to 20 carbon atoms, such as methyl, ethyl, n-propyl, isopropyl, n-butyl, isobutyl, t-butyl, pentyl, hexyl, heptyl, octyl, and the like. Examples of cycloalkyl groups include cyclopentyl and cyclohexyl.

The term “alkenyl” as used herein refers to a hydrocarbon group of 2 to 4, 2 to 8, or 2 to 20 carbon atoms and structural formula containing a carbon-carbon double bond.

The term “alkynyl” as used herein refers to a hydrocarbon group of 2 to 4, 2 to 8, or 2 to 20 carbon atoms and a structural formula containing a carbon-carbon triple bond.

The term “aryl” is defined as any carbon-based aromatic group including, but not limited to, phenyl, benzene, naphthalene, anthracene, phenanthrene, pyrene, and benzo[a]pyrene, etc.

The term “substituted aryl” is defined as an aryl group having at least one group attached to the aryl group that is not hydrogen. Examples of groups that can be attached to the aryl group include, but are not limited to, alkyl, alkynyl, alkenyl, aryl, heterocyclic, halide, nitro, amino, ester, ketone, aldehyde, hydroxy, carboxylic acid, alkoxy, cyano, alkoxy, thioalkyl, haloalkyl, hydroxyalkyl, alkylamino, diakylamino, or acyl. In various embodiments, a substituent is bound to carbon 2, 3, 4, 5, or 6 of one of these moieties. Examples of alkoxy substituents include, but are not limited to, methoxy, ethoxy, and isopropoxy groups. Examples of acyl substituents include acetyl and benzoyl groups.

The term “aralkyl” is defined as an aryl group having an alkyl, alkynyl, or alkenyl group attached to the aryl group. An example of an aralkyl group is a benzyl group.

The term “heteroaryl” is defined as an aryl group that has at least one heteroatom such as nitrogen, sulfur, or oxygen incorporated within the ring of the aryl group.

The term “heteroalkyl” is defined as an alkyl group that has at least one heteroatom, such as nitrogen, sulfur, oxygen, or phosphate, incorporated within the alkyl group or attached to the alkyl group.

### BRIEF DESCRIPTION OF THE DRAWINGS

FIG. 1 is a Western blot and a graph showing that buntanetap lowers APP in vitro in a dose-dependent manner in SH-SY-5Y human neuroblastoma cells.

FIG. 2A is a Table, collection of graphs and a Western blot showing that buntanetap treatment of APP/PS1 transgenic AD mice reduced APP and its fragments in hippocampus.

FIG. 2B shows dose-dependent improvement in cognition in the population with confirmed early AD.

FIG. 2C shows that Buntanetap's efficacy is strongly correlated with MMSE status.

FIG. 3 is a table showing the reduction of APP/Abeta, tau/phospho-tau and alpha-synuclein in the spinal fluid of mildly cognitive impaired patients.

FIGS. 4A-4B are graphs of the results of the electrophysiological study of Example 5.

FIGS. 4A and 4B show that impairment of synaptic plasticity in hippocampal slices APP/PS1 transgenic mice measured through Long-term potentiation (LTP) was prevented by treatment with buntanetap (1 mg/Kg) plus dulaglutide (0.1 mg/Kg). Treatment with the same concentrations of buntanetap (1 mg/Kg) and dulaglutide (0.1 mg/Kg) alone did not ameliorate the synaptic defect in the transgenic animals. FIG. 4A shows fEPSP of the last 20 minutes of the LTP curves.

FIGS. 5A-5C are graphs of the results of behavioral studies of Example 5.

FIG. 5A shows that impairment of short-term spatial memory in APP/PS1 transgenic mice measured through the 2-day radial arm water maze (RAWM) was prevented by treatment with Buntanetap (1 mg/Kg) plus dulaglutide (0.1 mg/Kg), whereas treatment with the same concentrations of Buntanetap (1 mg/Kg) and dulaglutide (0.1 mg/Kg) alone did not ameliorate the memory defect in the transgenic animals.

FIG. 5B shows the average number of errors during the last set of trials for the experiments shown in FIG. 5A.

FIG. 5C shows that impairment of associative memory in APP/PS1 transgenic mice as measured through contextual fear conditioning (FC) was prevented by treatment with Buntanetap (1 mg/Kg) plus dulaglutide (0.1 mg/Kg), whereas treatment with the same concentrations of Buntanetap (1 mg/Kg) and dulaglutide (0.1 mg/Kg) alone did not ameliorate the memory defect in the transgenic animals.

### DETAILED DESCRIPTION OF THE INVENTION

A protein called amyloid beta (“Abeta”) is infamous as a likely contributor to the development of Alzheimer's disease and dementia. Clumps of it, called plaques, are found throughout the brains of people who develop the cognitive symptoms of Alzheimer's disease. For unknown reasons, in a “sick” brain, the level of iron is high, and the synthesis of Abeta and other neurotoxic aggregating proteins is upregulated, leading to overproduction of Abeta and other neurotoxic aggregating proteins. High iron levels increase the translation of neurotoxic proteins, leading to impairment of axonal transport, inflammation, nerve cell death, and cognitive and motor impairments.

Abeta plaques are found throughout the brains of people who develop the cognitive symptoms of Alzheimer's.

Recent studies have revealed that diabetes mellitus is a risk factor for cognitive dysfunction or dementia, especially Alzheimer's disease (AD).

The invention is directed in part to a method to inhibit, prevent or treat, diabetes, metabolic syndrome, obesity, inflammation, oxidative stress, neurodegenerative disease (e.g., Alzheimer's disease) via the administration of buntanetap, compounds that are similar to buntanetap as described herein (e.g., a drug encompassing Formula (I), Formula (II), Formula (III) or Formula (IV)), pharmaceutically acceptable salts and complexes thereof, together with an appropriate antidiabetic agent, and one or more pharmaceutically acceptable excipients.

Buntanetap is a selective inhibitor of amyloid precursor protein (APP) production and has potential utility as a disease modifying treatment for AD (Cullen 2006; Utsuki 2006; Lahiri 2007). Buntanetap was discovered at the National Institute on Aging and was selected from a series of structurally related compounds designed for APP specificity with no or minimal acetylcholinesterase inhibitory activity. Buntanetap was shown to reduce APP and consequently beta-amyloid (A3) production in relevant preclinical in vitro and in vivo studies. Maccecchini, et al., “Buntanetap as a Candidate Drug to Lower CSF Amyloid Precursor Protein, Amyloid-β Peptide and τ Levels: Target Engagement, Tolerability and Pharmacokinetics in Humans”, J. Neurosurg. Psychiatry 2012; 83:894-902, hereby incorporated by reference, reported the results of a study of buntanetap single and multiple ascending dose phase 1 randomized, double blind, placebo-controlled safety, tolerance, pharmacokinetic studies were undertaken in 120 healthy human volunteers to define a dose that was then used in a small non-randomized study of five MCI subjects. Buntanetap doses up to 4×60 mg daily X 10 days were well tolerated. In plasma buntanetap, at all doses, was absorbed rapidly (Tmax=1.2 to 1.7 h) and cleared from the circulation biphasically (terminal half-life of 4.3-4.7 h). Buntanetap proved well tolerated and significantly lowered CSF levels of sAPPa, sAPPP, t-tau, p-tau, and specific inflammatory markers, and demonstrated a trend to lower CSF A34₂. Buntanetap's activity is also described in Applicant's U.S. Pat. No. 10,383,851, hereby incorporated by reference. Phase II data for Buntanetap has been published, C. Fang et al, Buntanetap, a Novel Translational Inhibitor of Multiple Neurotoxic Proteins, Proves to Be Safe and Promising in Both Alzheimer's and Parkinson 's Patients, J Prev Alzheimers Dis (2022). https://doi.org/10.14283/jpad.2022.84, published 10 Oct. 2022, hereby incorporated by reference in its entirety. This publication reported the results of a Phase 2a Clinical Study which was a double-blind, placebo-controlled, multi-center study of 14 early AD patients and 54 early PD patients. AD patients were given either 80 mg Buntanetap or placebo QD. PD patients were given 5 mg, 10 mg, 20 mg, 40 mg, 80 mg buntanetap or placebo QD. The primary endpoint was safety and tolerability; secondary endpoint is pharmacokinetics of Buntanetap in plasma. The Buntanetap was well tolerated at safe at doses up to 80 mg in both AD and PD patients. Cmax and AUC increased with dose without evidence for a plateau up to 80 mg QD. Biomarker data indicated a trend in lowering levels of neurotoxic proteins and inflammatory factors and improving axonal integrity ad synaptic function in both AD and PD cohorts. Psychometric tests showed statistically significant improvements in ADAS-Cog11 and WAIS coding in AD patients and MDS-UPDRS and WAIS coding in PD patients.

Buntanetap®, developed by QR Pharma, Inc. (now Annovis Bio, Inc.), is a small molecule that lowers soluble APP protein levels through a post-transcriptional mechanism. Buntanetap is also known as (+)-phenserine. Buntanetap is the stereoisomer of phenserine ((−)-N-phenylcarbamoyl eseroline), which reached clinical assessment for AD as an anticholinesterase inhibitor. Phenserine is an AChE inhibitor which has been investigated as being suitable as an agent for therapy for cognitive impairments associated with aging and Alzheimer's disease (U.S. Pat. No. 5,409,948). Due to its high cholinomimetic side effects, phenserine failed in 3 phase 3 clinical studies.

As used herein, the term “Buntanetap” refers to (3aR)-1,3a,8-trimethyl-1,2,3,3a,8,8a-hexahydropyrrolo[2,3-b]indol-5-yl phenylcarbamate, with the chemical structure shown in Formula IV below, at a chemical purity of at least 90%, preferably at least 95%, at least 98%, at least 99%, at least 99.5%, at least 99.9% or 100%, having the chemical structural as follows:

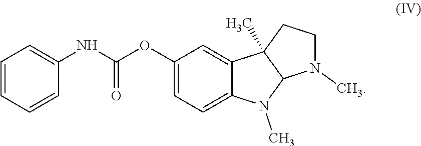 [SMILES: CN1CC[C@]2(C)c3cc(OC(=O)Nc4ccccc4)ccc3N(C)C12]

The term “chemical purity” as applied to (3aR)-1,3a,8-trimethyl-1,2,3,3a,8,8a-hexahydropyrrolo[2,3-b]indol-5-yl phenylcarbamate or a pharmaceutically acceptable salt of Buntanetap means the percent by weight of (3aR)-1,3a,8-trimethyl-1,2,3,3a,8,8a-hexahydropyrrolo[2,3-b]indol-5-yl phenylcarbamate or the pharmaceutically acceptable salt of Buntanetap in terms of (3aR)-1,3a,8-trimethyl-1,2,3,3a,8,8a-hexahydropyrrolo[2,3-b]indol-5-yl phenylcarbamate or the pharmaceutically acceptable salt of Buntanetap and other chemical impurities, e.g., its (−)-enantiomer, that may be present.

The invention also encompasses active metabolites of buntanetap. Active metabolites have previously been identified and include, for example, “N1-nor-Buntanetap” (which refers to (3aR)-3a,8-dimethyl-1,2,3,3a,8,8a-hexahydropyrrolo[2,3-b]indol-5-yl phenylcarbamate) or a salt thereof; “N8-nor-Buntanetap” (which refers to (3aR)-1,3a-dimethyl-1,2,3,3a,8,8a-hexahydropyrrolo[2,3-b]indol-5-yl phenylcarbamate) or a salt thereof, and “N1,N8-nor-Buntanetap” (which refers to (3aR)-3a-methyl-1,2,3,3a,8,8a-hexahydropyrrolo[2,3-b]indol-5-yl phenylcarbamate) or a salt thereof.

In other embodiments, the methods of the present invention are practiced using a phenserine or phenserine-like compound, metabolite, enantiomer, or derivative thereof, known to those skilled in the art, such as those described in U.S. Pat. Nos. 5,171,750; 6,410,747; 6,683,105, 7,153,882; 7,786,162; 7,973,057; 8,258,172; 8,546,430; 8,691,864; and 8,853,253, all of which are incorporated by reference in their entireties.

Diabetes mellitus is recognized as a group of heterogeneous disorders with the common elements of hyperglycemia and glucose intolerance due to insulin deficiency, impaired effectiveness of insulin action, or both. Diabetes is a chronic disease and there are three main types of diabetes type 1, type 2, and gestational diabetes (diabetes while pregnant). Type 1 diabetes is a condition wherein the pancreas doesn't make insulin or makes very little insulin. Insulin helps blood sugar enter the cells of a patient (e.g. a mammal such as a human) for use as energy. Without insulin, blood sugar can't get into cells and thus builds up in the bloodstream. High blood sugar is known to cause damage to the body. Type 2 diabetes is characterized by the cells of the body not responding normally to insulin (insulin resistance), thereby causing the pancreas to make more insulin in order to try to get the cells to respond. Eventually, the pancreas cannot keep up and the patient's blood sugar rises, setting the stage for prediabetes and type 2 diabetes. Prediabetes is a condition where blood sugar levels are higher than normal, but not high enough (as of yet) to be diagnosed as type 2 diabetes. More than 133 million Americans are dying with diabetes or prediabetes.

The prevalence of Type 2 diabetes mellitus increases with age and dementia also increases its incidence in later life. Recent studies have revealed that Type 2 diabetes mellitus is a risk factor for cognitive dysfunction or dementia, especially those related to Alzheimer's disease (AD). Insulin resistance, which is often associated with Type 2 diabetes mellitus, may induce a deficiency of insulin effects in the central nervous system (CNS). Insulin may have a neuroprotective role and may have some impact on acetylcholine (ACh) synthesis. Hyperinsulinemia, induced by insulin resistance occurring in Type 2 diabetes mellitus, may be associated with insulin deficiency caused by reduced insulin transport via the blood brain barrier (BBB). Insulin has multiple important functions in the brain. It is believed that insulin accelerates Alzheimer-related pathology through its effects on the amyloid beta (A3) metabolism and tau phosphorylation. Asymptomatic ischemic lesions in Type 2 diabetes mellitus subjects may lower the threshold for the development of dementia and this may explain the inconsistency between the basic research and clinicopathological studies.

In a broader sense, the invention is directed in part to the co-administration of a Buntanetap-type compound as defined by Formula (I), Formula (II), Formula (III) or Formula (IV) together with an antidiabetic agent.

The Buntanetap-type compounds include compounds having the Formula I or II as follows:

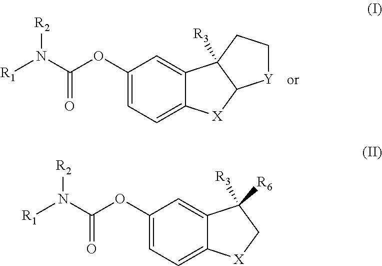

wherein R₁ and R₂ are, independently, hydrogen, branched or straight chain C₁-C₈ alkyl, substituted or unsubstituted aryl, heteroaryl, or aralkyl; R₃ is branched or straight chain C₁-C₄ alkyl or heteroalkyl or C₄-C₈ alkyl or heteroalkyl, or substituted or unsubstituted aryl; X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4, along with an effective amount of an (appropriate) antidiabetic agent.

The chiral center of compounds of Formula I and II is the carbon atom that has R₃ bonded to it. As depicted herein, the (+)-enantiomer has R₃ pointing behind the plane of the page. Although only the (+)-isomer is illustrated to save space, in other embodiments the compound having the Formula I or II can be the (+)-isomer, (−)-isomer, and mixtures of both isomers (e.g., racemic mixtures, including 1:1 racemic mixtures) of all of the compounds encompassed by the invention.

In certain embodiments, the compounds having the Formula I or II have an enantiomeric purity for the (+)-enantiomer of from 55 to 100%, desirably from 75 to 100%, more desirably from 85 to 100%, more desirably from 95 to 100%, and even more desirably 100%.

In certain preferred embodiments, wherein the compound having the Formula I or II is the substantially pure (+)-enantiomer.

In one embodiment, when the compound is Formula I, R₃ is methyl and X is NCH₃.

In one embodiment, when the compound is Formula I or II, R₃ is not methyl. In particular embodiments, R₃ is a branched or straight chain alkyl or heteroalkyl group of 2, 3, 4, 5, 6, 7, or 8 carbons or substituted or unsubstituted aryl.

In another embodiment, when the compound is Formula I or II, Y is C(H)R₄ or X is 0, S, or C(H)R₄.

In another embodiment, when the compound is Formula I, R₃ is methyl, X is NCH₃, and Y is NCH₃. In one embodiment, when the compound is Formula I, R₃ is methyl, X is NCH₃, Y is NCH₃, and R₁ is C₁-C₈ straight chain alkyl or benzyl and R₂ is hydrogen. In one embodiment, when the compound is Formula I, R₃ is methyl, X is NCH₃, Y is NCH₃, and R₁ is substituted or unsubstituted phenyl and R₂ is hydrogen. In one embodiment, when the compound is Formula I, R₃ is methyl, X is NCH₃, Y is NCH₃, and R₁ and R₂ are, independently, methyl or ethyl.

In another embodiment, when the compound is Formula I, R₃ is methyl, X is NCH₃, and Y is O. In one embodiment, when the compound is Formula I, R₃ is methyl, X is NCH₃, Y is 0, R₁ is C₁-C₈ straight chain alkyl or benzyl, and R₂ is hydrogen. In one embodiment, when the compound is Formula I, R₃ is methyl, X is NCH₃, Y is 0, and R₁ and R₂ are, independently, methyl or ethyl. In one embodiment, when the compound is Formula I, R₃ is methyl, X is NCH₃, Y is 0, and R₁ is substituted or unsubstituted phenyl and R₂ is hydrogen.

In another embodiment, when the compound is Formula I, R₃ is methyl, X is NCH₃, and Y is S. In one embodiment, when the compound is Formula I, R₃ is methyl, X is NCH₃, Y is S, R₁ is C₁-C₈ straight chain alkyl or benzyl, and R₂ is hydrogen. In one embodiment, when the compound is Formula I, R₃ is methyl, X is NCH₃, Y is S, and R₁ and R₂ are, independently, methyl or ethyl. In one embodiment, when the compound is Formula I, R₃ is methyl, X is NCH₃, Y is S, R₁ is substituted or unsubstituted phenyl, and R₂ is hydrogen.

In another embodiment, when the compound is Formula I, R₃ is methyl, X is NCH₃, and Y is NR₅. In one embodiment, when the compound is Formula I, R₃ is methyl, X is NCH₃, and Y is NR₅, wherein R₅ is —CH₂CH═CH₂, —CH₂CH₂Ph, benzyl, or hydrogen.

In another embodiment, when the compound has the Formula I, R₃ is methyl, Y is NCH₃, and X is NCH₃, wherein R₄ is benzyl or hydrogen.

In another embodiment, when the compound is Formula I, R₃ is methyl, X is NCH₃, Y is NR₅, wherein each R₄ and R₅ is, independently, hydrogen or benzyl.

In another embodiment, when the compound is Formula I, R₃ is phenyl, X is NCH₃, and Y is NCH₃.

In another embodiment, when the compound is Formula I, R₃ is methyl, and X is NCH₃, and Y is not NH or NHCH₂Ph.

In some embodiments, when the compound is Formula I, R₁ and R₂ are independently, hydrogen, substituted or unsubstituted aryl, R₃ is straight chain C₁-C₈ alkyl, X and Y are independently NR₅, wherein R₅ is independently hydrogen or straight chain C₁-C₈.

In some embodiments, when the compound is Formula I, R₁ and R₂ are independently, hydrogen or unsubstituted aryl, R₃ is straight chain C₁-C₈ alkyl, X and Y are independently NR₅, wherein R₅ is independently hydrogen or straight chain C₁-C₈.

In some embodiments, when the compound is Formula I, R₁ is hydrogen, R₂ is unsubstituted aryl, R₃ is methyl, X and Y are independently NR₅, wherein R₅ is independently hydrogen or methyl.

In a certain preferred embodiment, when the compound is Formula I, R₁ is hydrogen, R₂ is phenyl, R₃ is methyl, X is NCH₃, and Y is NCH₃.

In other embodiment, when the compound is Formula I, R₁ is hydrogen, R₂ is phenyl, R₃ is methyl, X is NCH₃, and Y is NH.

In a certain preferred embodiment, when the compound is Formula I, R₁ is hydrogen, R₂ is phenyl, R₃ is methyl, X is NH, and Y is NCH₃.

In a certain preferred embodiment, when the compound is Formula I, R₁ is hydrogen, R₂ is phenyl, R₃ is methyl, X is NH, and Y is NH.

In another embodiment, when the compound is Formula II, R₃ is methyl, X is C(H)CH₃, and R₆ is (CH₂)₂R₇, where R₇ is a substituted or unsubstituted amino group.

In a certain preferred embodiment, when the compound is Formula II, R₃ is methyl, X is NCH₃, and R₆ is (CH₂)₂R₇, where R₇ is a substituted or unsubstituted amino group.

In a certain preferred embodiment, wherein the compound having the Formula II is the substantially pure (+)-enantiomer.

The invention also relates to the use of a compound having the Formula (III) as follows:

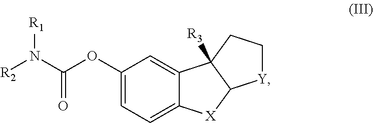

wherein R₁ and R₂ are, independently, hydrogen, branched or straight chain C₁-C₈ alkyl, substituted or unsubstituted aryl, heteroaryl, or aralkyl; R₃ is branched or straight chain C₁-C₄ alkyl or heteroalkyl or C₄-C₈ alkyl or heteroalkyl, or substituted or unsubstituted aryl; X is NR₅, wherein R₅ is C₂₋₈ alkenyl, C₂₋₈ alkynyl, or aralkyl, and Y is selected from C(H)R₄ or NR₅, wherein R₄ and R₅ are, independently, hydrogen, branched or straight chain C₁-s alkyl or heteroalkyl, alkenyl, or C₂-C₈ alkynyl, aralkyl.

As depicted herein, the (−)-enantiomer has R₃ pointing in front of the plane of the page. Although only the (−)-isomer is illustrated to save space, in other embodiments the compound having the Formula (III) can be the (+)-isomer, (−)-isomer, and mixtures of both isomers (e.g., racemic mixtures, including 1:1 racemic mixtures) of all of the compounds encompassed by the invention.

In a certain preferred embodiment, wherein the compound having the Formula (III) is the substantially pure (−)-enantiomer.

In one embodiment, when the compound is Formula (III), X is NR₅, wherein R₅ is aralkyl.

In one embodiment, when the compound is Formula (III), X and Y are NR₅, wherein R₅ is aralkyl.

In one embodiment, when the compound is Formula (III), wherein X is NR₅, wherein R₅ is aralkyl, and Y is NR₅, wherein R₅ is branched or straight chain C₁₋₈ alkyl or heteroalkyl.

In one embodiment, when the compound is Formula (III), wherein R₁ is branched or straight chain C₁-C₈ alkyl, aralkyl or aryl, R₂ is hydrogen, branched or straight chain C₁-C₈ alkyl, substituted or unsubstituted aryl, or aralkyl; Y is NR₅, wherein R₅ is aralkyl; and X is NR₅, wherein R₅ is hydrogen, branched or straight chain C₁₋₈ alkyl or heteroalkyl.

In one embodiment, when the compound is Formula (III), wherein R₁ is branched or straight chain C₁-C₈ alkyl, aralkyl or aryl; R₂ is hydrogen, branched or straight chain C₁-C₈ alkyl; Y is NR₅ where R₅ is benzyl; and X is NR₅, wherein R₅ is hydrogen, branched or straight chain C₁₋₈ alkyl or heteroalkyl.

In one embodiment, when the compound is Formula (III), wherein R₁ is branched or straight chain C₁-C₈ alkyl, aralkyl or aryl; R₂ is hydrogen, branched or straight chain C₁-C₈ alkyl; Y is NR₅ where R₅ is benzyl; and X is NR₅, wherein R₅ is hydrogen.

In one embodiment, when the compound is Formula (III), wherein R₁ is para-halophenyl; Y is NCH₃; and X is NR₅, wherein R₅ is alkyl or aralkyl, wherein R₁ is not para-phenyl bromophenyl when R₅ is benzyl.

In one embodiment, when the compound is Formula (III), wherein R₁ is para-isopropyl phenyl; R₂ is hydrogen; R₃ is methyl; Y is NR₅ where R₅ is benzyl; and X is NR₅, wherein R₅ is hydrogen.

Encompassed in the formulations of the invention are the (+)-isomer, (−)-isomer, and mixtures of both isomers (e.g., racemic 1:1 mixtures) of all of the compounds of the invention unless such compounds are specifically excluded.

Variables, such as R₁—R₇, n, X and Y throughout the application are the same variables as previously defined unless stated to the contrary.

The compounds described herein may form salts with acids or bases, and such salts are included in the present invention. In one embodiment, the salts are pharmaceutically acceptable salts. The term “salts” embraces addition salts of free acids or free bases that are compounds of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification, or formulation of compounds of the invention.

Examples of the pharmaceutically acceptable salt of buntanetap include acid addition salts prepared from a suitable acid. The suitable acid can be hydrobromic acid, hydrochloric acid, hydroiodic acid, sulfuric acid, carbonic acid, nitric acid, phosphoric acid, tetrafluoroboronic acid, perchloric acid, 1-hydroxy-2-naphthoic acid, 2,2-dichloroacetic acid, methanesulfonic acid, trifluoromethanesulfonic acid, 2-hydroxyethanesulfonic acid, benzenesulfonic acid, p-toluenesulfonic acid, cyclohexylaminosulfonic acid, 2-oxoglutaric acid, 4-acetamidobenzoic acid, acetic acid, phenylacetic acid, propionic acid, formic acid, succinic acid, glycolic acid, gluconic acid, malic acid, lactic acid, tartaric acid, citric acid, glucuronic acid, ascorbic acid, maleic acid, fumaric acid, pyruvic acid, aspartic acid, glutamic acid, benzoic acid, 4-hydroxybenzoic acid, anthranilic acid, 4-hydroxybenzoic acid, mandelic acid, pamoic acid, pantothenic acid, sulfanilic acid, stearic acid, alginic acid, O-hydroxybutyric acid, salicylic acid, galactaric acid and galacturonic acid. Preferably, the pharmaceutically acceptable salt is buntanetap tartrate, i.e., the acid addition salt of tartaric acid.

Suitable pharmaceutically acceptable base addition salts of compounds of the invention include, for example, metallic salts including alkali metal, alkaline earth metal and transition metal salts such as, for example, calcium, magnesium, potassium, sodium, and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N methylglucamine) and procaine. Examples of pharmaceutically unacceptable base addition salts include lithium salts and cyanate salts. All of these salts may be prepared from the corresponding compound by reacting, for example, the appropriate acid or base.

In certain preferred embodiments, the buntanetap compound(s) of the present invention is co-administered with an antidiabetic agent which is useful for treating type 1 diabetes, type 2 diabetes, or prediabetes.

In certain embodiments, the antidiabetic medication is insulin. Insulin is the most common type of medication used in type 1 diabetes treatment. There are more than 20 types sold in the United States. Insulin is administered as an infusion under the skin (with the help of an insulin pump) or as an injection. Insulin is generally administered via injection, and therefore may be combined with the co-administered compound (e.g., buntanetap) or administered separately. Further, the time of administration may be the same or different as insulin products have different durations of action than buntanetap. There are a number of factors when administering insulin and buntanetap, including but not limited to onset, peak levels, and duration of action. Onset is how long it takes before the insulins begin to work; peak levels are indicative of the time during which the insulins are at their maximum effectiveness in lowering blood glucose; and duration is how long the insulins continue to work before their effects wear off. Insulin helps control blood sugar levels in people whose bodies do not produce enough insulin. Insulin directly lowers glucose levels by increasing uptake into muscle and fatty tissue, and by reducing release of glucose from the liver. Insulin can be injected in the abdomen, legs, back of arms, or buttocks. Insulin may lead to weight gain.

Patients with type 1 diabetes (T1D) must be treated with insulin, as the beta cells in their pancreas no longer produce it. Insulin plays a vital role in glucose uptake and is required by the muscle and adipose tissue. However, insulin is not solely for patients with TlD; those with type 2 diabetes (T2D) may also be placed on insulin but generally only after failing to reach glycemic targets upon being placed on multiple oral agents for some time. Patients with diabetes typically receive multiple injections per day, including bolus insulin administered before meals and the long-acting basal insulin that lowers blood sugar levels over time. Insulin is classified as a high-risk drug because it can cause patients to experience hypoglycemia, but the benefits of this treatment surely outweigh the risks.

Glucagon-like peptide-1 receptor agonists (“GLP-1 RA” or “GLP-1 agonist”) are useful antidiabetic agents in the present invention, for the treatment of type 2 diabetes (T2D) because they effectively lower A1C and weight while having a low risk of hypoglycemia.

GLP-1 RAs increase glucose-dependent insulin secretion and decrease inappropriate glucagon secretion, delay gastric emptying, and increase satiety. There are currently at least eight approved GLP-1 receptor agonists: exenatide twice daily; exenatide once weekly; lixisenatide once daily; liraglutide once daily; exenatide once weekly; dulaglutide, once weekly; semaglutide once weekly, and oral semaglutide once daily.

Generally, long-acting agents result in greater A1C lowering than the short-acting agents, with semaglutide leading to the greatest A1C reduction. Out of the long-acting agents, exenatide XR appears to have the least impact on A1C, although it still produces more A1C lowering compared with the short-acting agents. In terms of A1C lowering, the agents could be ranked (from highest to lowest) in the following order: subcutaneous semaglutide >oral semaglutide >dulaglutide=liraglutide >exenatide XR >exenatide (twice daily)=lixisenatide.

Liraglutide (commercially available as Victoza® and Saxenda®) 18 mg/3 mL SC solution multidose pen; delivers doses of 0.6 mg, 1.2 mg, or 1.8 mg.

Lixisenatide (commercially available as Adlyxin® as a green pen starter dose of 50 mcg/mL in 3 mL prefilled pen (provides 14 doses of 20 mcg/dose) and as a burgundy pen (100 mcg/mL in 3 mL prefilled pen (provides 14 doses of 20 mcg/dose). In certain embodiments, the starting dose is 10 mcg SC qDay for 14 days, and the maintenance dose is achieved by increasing the dose to 20 mcg SC qDay starting on Day 15.

In certain embodiments, patients who are naive to basal insulin or GLP-1 agonists, currently on a GLP-1 receptor agonist, or on basal insulin <30 units/day may receive insulin glargine 15 units/lixisenatide 5 mcg SC qDay.

In certain embodiments, patients currently on basal insulin 30-60 units/day with or without a GLP-1 agonist may receive insulin glargine 30 units/lixisenatide 10 mcg SC qDay. (available as a 3-mL single-use pen (100 units/33 mcg) per mL)(Commercially available as Soliqua® 100/33)

Exenatide is commercially available under the tradename Byetta® as an injectable solution (prefilled pen) at 250 mcg/mL (1.2 mL vial) and 250 mcg/mL (2.4 mL vial). In certain embodiments, for adults, at first, 5 micrograms (mcg) may be injected under the skin 2 times a day at any time within the 60-minute period before the morning and evening meals (or before the 2 main meals of the day, about 6 hours or more apart). The dose may be adjusted after the first month of treatment to 10 mcg 2 times a day. Exenatide is also available as an extended-release suspension under the tradename Bydureon®, wherein, in certain embodiments, it is dosed in adults at 2 mg injected under the skin once every 7 days, at any time of the day, with or without meals.

Dulaglutide is available as an injection (pen) in 0.5 mL (0.75 mg, 1.5 mg, 3.0 mg, 4.5 mg), once a week. It is commercially available as Trulicity®. In certain embodiments, the recommended initiating dose of dulaglutide is 0.75 mg injected subcutaneously once weekly. If additional glycemic control is necessary, the dose may be increased to 1.5 mg once a week. If additional glycemic control is needed, the dose may be increased to 3 mg once a week after at least 4 weeks on the 1.5 mg dose.

Semaglutide is commercially available as Ozempic® once weekly semaglutide injection 0.5 mg, 1 mg, and 2 mg. In certain embodiments, the beginning dose is 0.25 mg once a week for the first 4 weeks. At Week 5, the dose may be increased to 0.5 mg once a week. The maximum dose of Ozempic® is 2.4 mg once a week subcutaneously. Semaglutide is also commercially available orally as Rybelsus®. In certain embodiments, semaglutide is orally administered initially as 3 mg PO qDay x30 days; the 3-mg dose is intended for treatment initiation and is not effective for glycemic control. After 30 days on 3 mg/day, the semaglutide dose may be increased to 7 mg PO qDay. After 30 days on 7 mg/day, the semaglutide dose may be increased to 14 mg PO qDay if additional glycemic control is needed.

Other useful classes of antidiabetic agents in the present invention are GLP-1/glucagon dual agonists. Such agents include cotadutide, pemvidutide (ALT-801). LY3298176, MEDI0382, and BI 456906 (dual glucagon-like peptide-1 and glucagon receptor agonists). Another useful class of antidiabetic agents include agents such as LY3437943 (a triple glucagon, GIP (glucose-dependent insulinotropic polypeptide) and GLP-1 receptor agonist), SAR441255 (a unimolecular peptide GLP-1/GIP/GCP receptor triagonist), LY2189265 (a long-acting glucagon-like peptide-1 analogue).

Another useful class of antidiabetic agent for the present invention is Tirzepatide (Mounjaro®) is the first drug in a new class of diabetes medications. It is a dual glucose-dependent insulinotropic polypeptide (GIP) and GLP-1 receptor agonist and, in certain embodiments, is administered as 5 mg, 10 mg or 15 mg subcutaneously once a week.

Another useful class of antidiabetic agent for the present invention is Glyxambi®, a combination of empagliflozin, a sodium-glucose cotransporter 2 (SGLT2) inhibitor and linagliptin, a dipeptidyl peptidase-4 (DPP-4) inhibitor. Jardiance® (empagliflozin) tablets combined with the only single-strength DPP-4i, Tradjenta® (linagliptin) tablets. In certain embodiments, the recommended dose of Glyxambi® is 10 mg empagliflozin and 5 mg linagliptin once daily, taken in the morning, with or without food. The dose may be increased to 25 mg empagliflozin and 5 mg linagliptin once daily. Sodium glucose cotransporter 2 (SGLT2) inhibitors are a newer, second line of medical treatment for the management of blood sugar levels in people with type 2 diabetes. SGLT2 inhibitors work by blocking the reabsorption of glucose from the kidneys back into the bloodstream. Examples of SGLT2 inhibitors include canagliflozin (Invokana@), canagliflozin-metformin (Invokamet®, Invokamet® XR), dapagliflozin (Farxiga®), dapagliflozin-metformin (Xigduo® XR), dapagliflozin-saxagliptin (Qtern®), empagliflozin (Jardiance®), empagliflozin-linagliptin (Glyxambi®), empaglifozin-linagliptin-metmorfin (Trijardy®XR), empagliflozin-metformin (Synjardy®, Synjardy® XR), and ertugliflozin (Steglatro®).

Also useful as antidiabetic agents for co-administration with the buntanetap-type drugs in the present invention include biguanides such as metformin; sulfonylureas such as glipizide, glimepriride (Amaryl®), glimepiride-pioglitazone (Duetact®), gliclazide, glipizide ER (Glipizide® XL, Glucotrol® XL), glipizide-metformin, glyburide (Glynase®), glyburide-metformin; sodium glucose cotransporter 2 inhibitor class agents such as Invokana®; dipeptidyl peptidase 4 inhibitors such as Januvia®; SGLT2 class agents such as Jardiance; glucagon-like peptide 1 agonists such as Victoza®, and other glucagon-like peptide 1 agonists such as Trulicity®; alpha-glucosidase inhibitors such as acarbose and miglitol (Glyset®); dopamine-2 agonists such as bromocriptine (Clycloset®, Parlodel®); meglitinides (nateglinide (Starlix®, repaglinide (Prandin®)(which aid in the release of insulin); thiazolidinediones (which work by decreasing glucose in the liver; examples are rosiglitazone, pioglitazone-alogliptin (Oseni®), pioglitazone-glimepiride (Duetact®), and pioglitazone-metformin (Actoplus® Met, Actoplus® Met XR).

Another useful class of antidiabetic agent for the present invention is DPP-4 inhibitors. Examples of DPP-4 inhibitors include alogliptin (Nesina®), alogliptin-metformin (Kazano®), linagliptin (Tradjenta®), linagliptin-empagliflozin (Glyxambi®), linagliptin-metformin (Jentadueto®, Jentadueto® XR), saxagliptin (Onglyza®), saxagliptin-metformin (Kombiglyze® XR), sitagliptin (Januvia®), sitagliptin-metformin (Janumet® and Janumet® XR), sitagliptin and simvastatin (Juvisync®).

Another useful class of antidiabetic agent for the present invention is metformin, which is considered the first-line oral agent for patients with diabetes and can be used to treat pre-diabetes. It works by decreasing glucose production in the liver, increasing insulin sensitivity, and lowering intestinal sugar absorption. In certain embodiments, metformin (Fortamet®) is administered as 1000 milligrams (mg) once-a-day taken with the evening meal, which may be increased if needed until the blood sugar of the patient is controlled. However, the dose is usually not more than 2500 mg per day. In certain embodiments, metformin alone in extended-release form (e.g., Glucophage® XR) may be administered at first as 500 mg once daily with the evening meal, which may be increased if needed until the patient's blood sugar is controlled. However, the dose is usually not more than 2000 mg per day.

In certain embodiments, metformin may be administered with a sulfonylurea. Metformin may also be administered with insulin, at first as a 500 mg once-a-day dose, which may be increased by 500 mg every week if needed until the patient's blood sugar is controlled. However, the dose is usually not more than 2500 mg per day. Metformin can also be combined with other type 2 diabetes medications, and is found in the following medications: metformin-alogliptin (Kazano®), metformin-canagliflozin (Invokamet®), metformin-dapagliflozin (Xigduo® XR), metformin-empagliflozin (Synjardy®), metmorfin-ertugliflozin (Segluromet®), metformin-glipizide, metformin-glyburide (Glucovance®), metformin-linagliptin (Jentadueto®, Jentadueto® XR), metformin-pioglitazone (Actoplus® Met, Actoplus® Met XR), metformin-repaglinide (PrandiMet®), metformin-rosiglitazone (Avandamet®), metformin-saxagliptin (Kombiglyze® XR), and metformin-sitagliptin (Janumet®, Janumet® XR).

In certain embodiments, metformin may be administered as an oral extended-release suspension. For adults, that dose is at first 5 milliliters (mL) once-a-day taken with the evening meal, which may be increased by 5 mL weekly if needed until the patient's blood sugar is controlled. However, the dose is usually not more than 20 mL per day.

Metformin may also be administered as an oral solution. At first, 5 milliliters (mL) of metformin solution (alone) may be administered two times a day, or 8.5 mL once-a-day with meals. This may be increased if needed until the blood sugar is controlled. However, the dose is usually not more than 25.5 mL per day.

Metformin may also be administered with insulin, at first, as 5 mL once-a-day and increased if needed until the blood sugar is controlled. However, the dose is usually not more than 25 mL per day.

In certain embodiments, the antidiabetic agent is insulin. The most common insulins are Basaglar® (long-acting) and NovoLog® (rapid-acting). Regular or “short-acting” insulin may reach the bloodstream 30 minutes after injection and peak 2-3 hours afterward. These injections also work up to 3-6 hours. Types of regular insulin include: Humulin® R U-100, Novolin® R FlexPen, Novolin® R ReliOn, Novolin® R FlexPen ReliOn.

Rapid-acting insulins work within 15 minutes. The peak time is 1 to 2 hours after use, and the medication lasts between 2 and 4 hours. Available types of rapid-acting insulin include inhaled insulin (Afrezza®); insulin aspart (Fiasp®, Fiasp® FlexTouch, Fiasp® PenFill, NovoLog®, NovoLog® FlexPen, NovoLog® FlexTouch, NovoLog® PenFill, ReliOn NovoLog®, ReliOn NovoLog® FlexPen); insulin glulisine (Apidra®, Apidra® SoloStar), which is only available as a brand-name drug insulin lispro (Admelog®, Admelog® SoloStar, Humalog®, Humalog® KwikPen, Humalog® Junior KwikPen), insulin lispro-aabc (Lyumjev®, Lyumjev® KwikPen).

Intermediate-acting insulins work about 2-4 hours after use, with an average peak time of 12 hours. They generally last between 12 and 18 hours. Examples include: insulin isophane (Humulin® N U-100, Humulin® N KwikPen, Novolin® N, Novolin® N FlexPen, Novolin® N ReliOn, Novolin® N FlexPen ReliOn).

Long-acting insulins lower blood glucose levels for up to 24 hours or longer and reach the bloodstream more gradually. Available long-acting insulin include: insulin degludec (Tresiba®, Tresiba® FlexTouch), insulin detemir (Levemir®), insulin glargine (Basaglar® KwikPen, Lantus, Lantus® SoloStar, Toujeo® SoloStar, Toujeo® Max SoloStar), insulin glargine-yfgn (Semglee-yfgn®) concentrated regular insulin (Humulin® R U-500, Humulin® R U-500 KwikPen).

Combination (premixed) insulins include insulin aspart protamine/insulin aspart 70/30 (NovoLog® Mix 70/30, NovoLog® Mix 70/30 FlexPen), insulin isophane/regular insulin 70/30 (Humulin® 70/30, Humulin® 70/30 KwikPen, Novolin® 70/30, Novolin® 70/30 FlexPen, Novolin® 70/30 FlexPen ReliOn), insulin lispro protamine/insulin lispro 50/50 (Humalog® Mix 50/50, Humalog Mix 50/50 KwikPen), insulin lispro protamine/insulin lispro 75/25 (Humalog® Mix 75/25, Humalog® Mix 75/25 KwikPen).

Another useful class of antidiabetic agent for the present invention is Pramlintide (SymlinPen®), which is an amylinomimetic. It is an injectable used before meals, and delays the time the stomach takes to empty itself as well as reducing the secretion of glucagon after meals (resulting in a lowered blood sugar).

In addition to the above, examples of suitable insulins for the present invention include but are not limited to Insulin glulisine (commercially available as Apidra®) having an onset of 5-15 minutes, a peak concentration which occurs 1-3 hours after administration, and a duration of 3-5 hours; Insulin aspart (commercially available as Novolog®) having an onset of 10-15 minutes, a peak concentration which occurs 1-3 hours after administration, and a duration of 3-5 hours; Insulin lispro U-100/U-200 (commercially available as Humalog®) having an onset of 10-15 minutes, a peak concentration which occurs 1-3 hours after administration, and a duration of 3-5 hours; Regular insulin (commercially available as Novolin R, Humulin R) having an onset of 30-60 minutes, a peak concentration which occurs 2-4 hours after administration, and a duration of 5-8 hours; NPH insulin (commercially available as Nololin N, Humulin N) having an onset of 1-2 minutes, a peak concentration which occurs 4-12 hours after administration, and a duration of 14-24 hours; Insulin detemir (commercially available as Levemir®) having an onset of about 1 hour, a peak concentration which occurs 3-14 hours after administration, and a duration of up to about 24 hours; Insulin U-100 (commercially available as Lantus®, Basaglar®) having an onset of 3-4 hours, no peak concentration, and a duration of up to about 24 hours; Insulin glargine U-300 (commercially available as Toujeo®) having an onset of about 6 hours after administration, no peak, and a duration of up to about 36 hours; Insulin degludec U-100/U-200 (commercially available as Tresiba®) having an onset of about 1 hour, no peak concentration, and a duration of up to about 42 hours; Pre-mixed insulin (commercially available as Humalog® mix 75/25 (75% NPL and 25% insulin Lispro)) having an onset of 5-20 minutes, a peak concentration which occurs 1-2 hours after administration, and a duration of 10-16 hours; Pre-mixed insulin 50/50 (50% insulin lispro protamine and 50% insulin lispro) having an onset of 5-20 minutes, a peak concentration which occurs 1-2 hours after administration, and a duration of 10-16 hours; Novolog® mix 70/30 (70% insulin aspart protamine and 30% insulin aspart) having an onset of 5-20 minutes, a peak concentration which occurs 1-2 hours after administration, and a duration of 10-16 hours; and the like. This list is not meant to be all-inclusive, but rather provides examples of useful insulin products.

Humalog®, Novolog®, and Apidra® may be taken up to 15 minutes before eating and, if using vials, may be mixed with NPH. Regular insulin is taken 30 minutes before eating and also may be mixed with NPH. NPH (Neutral Protamine Hagedorn) is a longer acting human insulin that is used to cover blood sugar between means and to satisfy overnight insulin requirements. Insulins made by different manufacturers should not be mixed, e.g., Humulin-R® (Eli Lilly) should not be mixed with Novolin-N® (Novo-Nordisk). Manufacturers use different processes to produce their insulins, and different preservatives. Their interactions have not been studied. Side effects of insulin administration include hypoglycemia (low blood sugar) and local allergic reaction (rare).

Examples of suitable insulins which can be co-administered when the patient is a type 1 diabetic include but are not limited to Insulin glulisine (commercially available as Apidra®) having an onset of 5-15 minutes, a peak concentration which occurs 1-3 hours after administration, and a duration of 3-5 hours; Insulin aspart (commercially available as Novolog®) having an onset of 10-15 minutes, a peak concentration which occurs 1-3 hours after administration, and a duration of 3-5 hours; Insulin lispro U-100/U-200 (commercially available as Humalog®) having an onset of 10-15 minutes, a peak concentration which occurs 1-3 hours after administration, and a duration of 3-5 hours; Regular insulin (commercially available as Novolin R, Humulin R) having an onset of 30-60 minutes, a peak concentration which occurs 2-4 hours after administration, and a duration of 5-8 hours; NPH insulin (commercially available as Nololin N, Humulin N) having an onset of 1-2 minutes, a peak concentration which occurs 4-12 hours after administration, and a duration of 14-24 hours; Insulin detemir (commercially available as Levemir®) having an onset of about 1 hour, a peak concentration which occurs 3-14 hours after administration, and a duration of up to about 24 hours; Insulin U-100 (commercially available as Lantus®, Basaglar®) having an onset of 3-4 hours, no peak concentration, and a duration of up to about 24 hours; Insulin glargine U-300 (commercially available as Toujeo®) having an onset of about 6 hours after administration, no peak, and a duration of up to about 36 hours; Insulin degludec U-100/U-200 (commercially available as Tresiba®) having an onset of about 1 hour, no peak concentration, and a duration of up to about 42 hours; Pre-mixed insulin (commercially available as Humalog® mix 75/25 (75% NPL and 25% insulin Lispro)) having an onset of 5-20 minutes, a peak concentration which occurs 1-2 hours after administration, and a duration of 10-16 hours; Pre-mixed insulin 50/50 (50% insulin lispro protamine and 50% insulin lispro) having an onset of 5-20 minutes, a peak concentration which occurs 1-2 hours after administration, and a duration of 10-16 hours; Novolog® mix 70/30 (70% insulin aspart protamine and 30% insulin aspart) having an onset of 5-20 minutes, a peak concentration which occurs 1-2 hours after administration, and a duration of 10-16 hours; and the like. This list is not meant to be all-inclusive, but rather provides examples of useful insulin products.

Humalog®, Novolog®, and Apidra® may be taken up to 15 minutes before eating and, if using vials, may be mixed with NPH. Regular insulin is taken 30 minutes before eating and also may be mixed with NPH. NPH (Neutral Protamine Hagedorn) is a longer acting human insulin that is used to cover blood sugar between means and to satisfy overnight insulin requirements. Insulins made by different manufacturers should not be mixed, e.g., Humulin-R® (Eli Lilly) should not be mixed with Novolin-N® (Novo-Nordisk). Manufacturers use different processes to produce their insulins, and different preservatives. Their interactions have not been studied. Side effects of insulin administration include hypoglycemia (low blood sugar) and local allergic reaction (rare).

Another useful class of antidiabetic agent for the present invention is androstenetriol and similar compounds, which include, e.g., androst-5-ene-3β,7β,17β-triol (β-androstenetriol, or βAET) and synthetic analogues of βAET (e.g., NE3107 and HE3286).

βAET is a steroid produced in the adrenal glands as a metabolite of DHEA. It is believed to have similar effects to DHEA and androstenediol. The compound is a weak androgen and estrogen, but does not attach to the androgen, estrogen, progesterone, or glucocorticoid receptors.

HE3286 and NE3107 are synthetic analogues of βAET.

NE3107 is a derivative of β-androstenetriol, a naturally occurring adrenal sterol metabolite. As compared to the parent compound, NE3107 has been chemically modified to increase oral bioavailability and stability. It has anti-inflammatory and insulin-sensitizing actions and enters the brain. NE3107 binds to ERK1/2, kinases involved in inflammatory signaling and insulin responses.

Another useful class of antidiabetic agent for the present invention is a PPAR activator, which include, e.g., T3D-959, pioglitazone, daidzein, astaxanthin, icariin, glabridin, gemfibrozil, CDDO-Im, palmitelaidic acid, alpinetin, ophiopogonin D, oroxin A, cloxiquine, bilobetin, licarin B, Falcarindiol, CUDA, adelmidrol, (S)-Coriolic acid, daidzein, and Gypenoside XLIX.

In certain embodiments, the antidiabetic agent is T3D-959. T3D-959 regulates the genes involved in glucose and fat metabolism, or energy production, a process associated with Alzheimer's disease risk, and is a dual agonist of the peroxisome proliferator activated nuclear receptor delta/gamma, aka PPAR6/y. T3D-959 activates PPAR delta and PPAR gamma, which are two nuclear receptors that are central regulators of normal glucose and lipid metabolism in the brain. Both PPAR delta and PPAR gamma targets are central regulators of glucose and lipid metabolism. Agonism (activation) of both receptors maintains metabolic homeostasis in similar and different ways. PPAR delta is highly expressed throughout the central nervous system (CNS) and enriched in areas of the brain involved in energy homeostasis, e.g. the mediobasal hypothalamus. It is being developed as an oral therapy for Alzheimer's disease in doses of 15 mg, 30 mg, and 45 mg. In the methods of the invention, T3D-959 may, e.g., be administered orally once-a-day.

“Type 3 diabetes” is a term some people use to describe Alzheimer's disease. Some scientists proposed the term because they believe insulin dysregulation in the brain causes dementia. However, type 3 diabetes is not an officially recognized health condition. Authors of a 2008 review, S. de la Monte, Alzheimer's Disease Is Type 3 Diabetes-Evidence Reviewed, J Diabetes Sci Technol v.2(6); 2008 November, conclude that the term “type 3 diabetes” accurately reflects the fact that Alzheimer's disease is a form of diabetes that affects the brain. They used human and animal studies to explain how this happens.

The review describes Alzheimer's disease as a neuroendocrine disease involving impaired insulin and insulin-like growth factor (IGF) signaling. The condition can also involve inflammation and oxidative stress. The authors note that while obesity and Type 2 Diabetes might contribute to the development of Alzheimer's disease, they are not sufficient causes by themselves. However, a more recent study, K. Mittal, et al., Type 3 Diabetes: Cross Talk between Differentially Regulated Proteins of Type 2 Diabetes Mellitus and Alzheimer 's Disease, Scientific Reports volume 6, Article number: 25589 (2016) suggests that insulin-degrading enzyme may shift type 2 diabetes to type 3 diabetes by altering metabolic pathways. This process may result in oxidative stress and beta-amyloid in the brain, which are both characteristics of Alzheimer's disease.

A 2020 review, T. Hguyen et al., Type 3 Diabetes and Its Role Implications in Alzheimer's Disease, Int J Mol Sci. 2020 May; 21(9): 3165 lists the potential risk factors for developing type 3 diabetes. These include a diet high in calories, sugar, and fat and low in fiber, low socioeconomic status, exposure to stress, race and ethnicity, lack of physical activity, genetics, family history, birth weight. The same study indicates that high blood pressure and impaired lipid, or fat, transportation play a role in the development of Alzheimer's disease.

Additionally, others have concluded that having the APOE4 gene can increase a person's risk of the condition. N. Zhao, et al., Apolipoprotein E4 Impairs Neuronal Insulin Signaling by Trapping Insulin Receptor in the Endosomes, J. Neuron, Article, Volume 96, Issue 1, P115-129E.5, Sep. 27, 2017; DOI:https://doi.org/10.1016/j.neuron.2017.09.003.

### Administration and Dosing

In the methods of the invention, buntanetap, its analogs, metabolites, or pharmaceutically acceptable salts thereof and the antidiabetic agent can be administered parenterally or enterally. Examples of the route of administration of buntanetap, or an analog, metabolite, or pharmaceutically acceptable salts thereof, and the antidiabetic agent are intravenous, intraocular, intramuscular, subcutaneous, topical, oral, sublingual, and buccal. Preferably, for purposes of the present invention, buntanetap is administered orally.

In the present invention, buntanetap, or a pharmaceutically acceptable salt of buntanetap, can be administered once, twice, three times, or four times daily. Buntanetap is preferably administered on a once-a-day basis. Depending on the route of administration, buntanetap is administered in different dose ranges.

In certain embodiments buntanetap is administered orally in an amount from about 0.01 mg to about 120 mg, preferably on a once-a-day basis. In certain preferred embodiments, buntanetap is administered in an amount from about 0.01 mg to about 10 mg, from about 0.01 mg to about 5 mg, from about 0.01 mg to about 0.9 mg, or from 0.01 mg to about 0.8 mg. preferably on a once-a-day basis.

In certain embodiments, buntanetap or a pharmaceutically acceptable salt thereof is administered (i) orally in an amount from about 0.1 mg to about 5 mg on a once-a-day, twice-a-day, three times a day, or four times a day basis; (ii) intravenously in an amount from about 0.01 mg to about 0.3 mg/day; or (ii) intraperitoneally/intramuscularly (IP/IM) in a dose from about 0.03 to about 7 mg/day.

In certain embodiments, buntanetap or a pharmaceutically acceptable salt thereof is administered (i) orally in an amount from about 0.1 mg to about 5 mg on a once-a-day, twice-a-day, three times a day, or four times a day basis; (ii) intravenously in an amount from about 0.01 mg to about 0.3 mg/day; or (ii) intraperitoneally/intramuscularly (IP/IM) in a dose from about 0.03 to about 0.7 mg/day.

In certain embodiments, buntanetap or a pharmaceutically acceptable salt thereof is administered (i) orally in an amount from about 0.01 mg to about 10 mg, from about 0.05 mg to about 5 mg or from about 0.1 mg to about 3 mg on a once-a-day, twice-a-day, three times a day, or four times a day basis; (ii) intravenously in an amount from about 0.01 mg to about 0.3 mg/day; or (ii) intraperitoneally/intramuscularly (IP/IM) in a dose from about 0.03 to about 7 mg/day.

In certain embodiments, the compounds are administered in accordance with the formulations and methods of the invention to a patient who is already presenting with symptoms of a neurological disease. In other embodiments, the compounds are administered in accordance with the formulations and methods of the invention to a patient who is already presenting with pulmonary hypertension. In yet other embodiments, the compounds are administered in accordance with the formulations and methods of the invention to a patient (human subject) who is not presenting with any neurological or hypertensive problems. In certain preferred embodiments, buntanetap is administered orally in a dose from about 0.01 mg to about 5 mg. In other embodiments, the buntanetap dose is administered intravenously in an amount from about 0.01 to about 3 mg/day. In other preferred embodiments, the buntanetap dose is administered intraperitoneally/intramuscularly (IP/IM) in a dose from about 0.03 to about 7 mg/day.

In certain embodiments of each of the methods of the present invention as described above, the oral pharmaceutical composition includes from about 0.01 mg to about 10 mg, from about 0.05 mg to about 5 mg, from about 0.1 mg to about 5 mg, from about 0.25 mg to about 5 mg, or from about 0.1 mg to about 0.8 mg buntanetap or a pharmaceutically acceptable salt thereof, the IP/IM pharmaceutical composition includes from about 0.03 to about 7 mg buntanetap or a pharmaceutically acceptable salt thereof, and the intravenous (IV) pharmaceutical formulation includes from about 0.01 to about 3 mg buntanetap or a pharmaceutically acceptable salt thereof.

In general, the dose of buntanetap preferred to be administered to healthy human patients is a tolerable dose, i.e., a dose that does not cause untoward side effects in a majority of human patient, which dose is also effective for prophylactic treatment of the healthy human(s) with respect to, e.g., neurodegenerative diseases, cancer, cardiovascular homeostasis, diseases or conditions of vital organs, cardiovascular disease, and the like.

In certain preferred embodiments of the methods described herein, peak plasma circulating levels of buntanetap in humans range, e.g., from about 0.01 ng/mL to about 40 ng/mL, in certain embodiments from about 0.2 ng/mL to about 2 ng/mL, and more preferably from about 0.3 ng/mL to about 12 ng/mL. In certain preferred embodiments, the peak plasma circulating level is reached within about 6 hours after administration of buntanetap to humans.

In certain embodiments, the peak plasma circulating level is reached within about 3 hours after administration of buntanetap to the humans. In certain embodiments, the plasma circulating level of buntanetap is equal to or greater than about 0.01 ng/mL, 0.05 ng/mL, 0.1 ng/mL, 0.2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 mg/mL, 15 ng/mL, 16 ng/mL, 17 ng/mL, 18 ng/mL, 19 ng/mL, or 20 ng/mL for at least 9 hours, and preferably for at least 12 hours, after administration of buntanetap to humans. In certain embodiments, the half-life of buntanetap in cerebrospinal fluid after administering is about 12 hours, and the half-life of buntanetap in plasma after administering is about 5 hours.

The therapeutic agent(s) used as the buntanetap-type drug and antidiabetic agents in the formulations and treatments of the present invention are preferably dosed in therapeutically effective amounts known to those skilled in the art or disclosed herein. In certain embodiments, the therapeutically effective amount is an amount that yields a maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. In other embodiments, the therapeutic amount of the antidiabetic agent or the T3D drug is subtherapeutic, as compared to that drug being administered without the buntanetap-type drug. One skilled in the clinical and pharmacological arts will be able to determine a therapeutically effective amount through routine experimentation, namely by monitoring a subject's response to administration of the agent and adjusting the dosage accordingly. In certain embodiments, the antidiabetic agent(s) is administered to the patent at appropriate time intervals (e.g., concurrently with the buntanetap) and via an appropriate route of administration (e.g., oral, subcutaneous, intravenous, intramuscular). In certain embodiments, the antidiabetic agent(s) is administered together with the buntanetap-type drug in a single formulation where possible).

### Pharmaceutical Compositions and Therapies

Administration of compounds useful within the invention may be achieved in a number of different ways, using methods known in the art. The therapeutic and prophylactic methods of the invention thus encompass the use of pharmaceutical compositions comprising the compounds useful within the invention to practice the methods of the invention.

The relative amounts of the active ingredients, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Although the description of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as, e.g., non-human primates, cattle, pigs, horses, sheep, camels, elephants, cats, and dogs.

In certain embodiments, buntanetap dosages which may be administered in a method of the invention to an animal, preferably a human, range in amount from 0.05 g to about 50 mg per kilogram of body weight of the animal. While the precise dosage administered will vary depending upon any number of factors, including but not limited to, the type of animal and type of disease state being treated, the age of the animal and the route of administration, the dosage of the compound will preferably vary from about 1 g to about 10 mg per kilogram of body weight of the animal. In certain embodiments, the dosage will vary from about 3 g to about 30 mg per kilogram of body weight of the animal.

Pharmaceutical compositions that are useful in the methods of invention may be prepared, packaged, or sold in formulations suitable for oral, parenteral, topical, buccal, or another route of administration. Other contemplated formulations include projected nanoparticles, liposomal preparations, resealed erythrocytes containing the active ingredient, and immunologically based formulations.

A pharmaceutical composition of the invention may be prepared, packaged, or sold in bulk, as a single unit dose, or as a plurality of single unit doses of, e.g., the buntanetap and the antidiabetic agent. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The composition of the invention may consist of the active ingredient alone, in a form suitable for administration to a (human) subject or patient, or the composition may comprise at least one active ingredient and one or more pharmaceutically acceptable excipients.

In one embodiment, the compositions of the invention are formulated using one or more pharmaceutically acceptable carriers. Pharmaceutically acceptable carriers that are useful, include, but are not limited to, glycerol, water, saline, ethanol, and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey). The carrier may be a solvent or dispersion medium containing, for example, water, ethanol, polyol (for example, glycerol, propylene glycol, and liquid polyethylene glycol, and the like), suitable mixtures thereof, and vegetable oils.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition may include an antioxidant and a chelating agent that inhibits the degradation of the compound. Preferred antioxidants for some compounds are BHT, BHA, alpha-tocopherol, and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing, or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

Controlled- or sustained-release formulations of a composition of the invention may be made using conventional technology, in addition to the disclosure set forth elsewhere herein. In some cases, the dosage forms to be used can be provided as slow or controlled release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the compositions of the invention.

Controlled release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, nanoparticles, or microspheres or a combination thereof that facilitates the controlled-release of the active ingredient.

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans-, and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

For oral administration, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more inert, non-toxic pharmaceutically excipients. Such excipients include, for example an inert diluent such as lactose; granulating and disintegrating agents such as cornstarch; binding agents such as starch; and lubricating agents such as magnesium stearate. The oral compositions of the invention in the form of tablets or capsules prepared by conventional means with pharmaceutically acceptable excipients such as binding agents; fillers; lubricants; disintegrates; or wetting agents.

Tablets may be non-coated, or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation. For oral administration, if desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400).

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin. Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid preparation for oral administration may be in the form of solutions, syrups, or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters, or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen free water) prior to parenteral administration of the reconstituted composition.

A pharmaceutical composition of the invention may be prepared, packaged, or sold in a formulation suitable for topical administration. There are several advantages to delivering compounds, including drugs or other therapeutic agents, into the skin (dermal drug delivery) or into the body through the skin (transdermal drug delivery). Transdermal compound delivery offers an attractive alternative to injections and oral medications.

Additional dosage forms of this invention include dosage forms as described in U.S. Pat. Nos. 6,340,475; 6,488,962; 6,451,808; 5,972,389; 5,582,837 and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### DETAILED DESCRIPTION OF PREFERRED EMBODIMENTS

The following examples further illustrate aspects of the present invention. They are provided for the purpose of illustration only, and the invention is not limited to these examples, but rather encompasses all variations that are evident as a result of the teachings provided herein.

### Example 1

Three Phase I clinical studies have established buntanetap's safety. The pharmacokinetic analyses have demonstrated that the small lipophilic molecule readily enters the brain where its concentration is about 8 times higher than in plasma. Importantly, buntanetap normalized levels of APP, Tau, and aSYN in the cerebrospinal fluid (CSF) of MCI subjects at a dose of 4×60 mg/day. Buntanetap had a>12 h half-life in CSF of MCI subjects, and its effect in lowering these neurotoxic proteins and inflammation extended throughout the 12 h sampling period after the last dose (Maccecchini, et al., “Buntanetap (posiphen) as a Candidate Drug to Lower CSF Amyloid Precursor Protein, Amyloid-βPeptide and τ Levels: Target Engagement, Tolerability and Pharmacokinetics in Humans”, J. Neurosurg. Psychiatry 2012; 83:894-902). Therefore, we conclude that a much lower single daily buntanetap dose would be effective in the proposed study. In fact, much lower doses were studied in a double Alzheimer/Parkinson phase 2 study that was conducted and completed in 2021(effective IND #72,654). The double phase 2 clinical trial recruited 14 AD (Alzheimer's disease) and 54 patients and treated them over 25 with a once daily dose of buntanetap. The 14 AD patients receive either 80 mg QD or placebo, whereas the 54 PD (Parkinson's disease) patients received 5, 10 20, 40 80 mg QD or placebo. In a nutshell the data shows that in AD and in PD patients (a) buntanetap crossed the blood brain barrier, (b) reduced neurotoxic protein biomarkers, (c) reduced inflammatory markers, (d) improved axonal and synaptic function, and most importantly improved the affected function in both patient populations. In AD patients, buntanetap improved cognition as measured by ADAS-Cog11 and WAIS coding speed (achieving statistical significance versus baseline at 80 mg dose but not placebo). In PD patients it improved at all doses motor function as measured by MDS-UPDRS (Part II, III, IV and total) with the maximum improvement for 10 & 20 mg and improved WAIS speed and accuracy (achieving statistical significance versus placebo in the 5 mg, 20 mg and 80 mg dose arms [p<0.05] of the broader study population [n=54] with the total for all doses also reporting statistically significant improvement [p<0.001]). The data demonstrates the potential benefits of reducing the overexpression of neurotoxic aggregating proteins on inflammation, axonal and synaptic function, and cognitive and functional health. We expect a larger sample population will allow buntanetap to fully demonstrate statistically significant cognitive and functional improvement resulting from the normalization of toxic protein levels in the next planned AD study as has already been demonstrated for PD (Fang et al., Prev Alz Dis 2022).

Buntanetap's effect on neurotoxic proteins. The drug lowers levels of APP in vitro in neuroblastoma cells (Mikillineni et al: Parkinson's Disease; Volume 2012, Article ID 142372, 13 pages. The Anticholinesterase Phenserine and Its Enantiomer Buntanetap as 5′ Untranslated-Region-Directed Translation Blockers of the Parkinson's Alpha Synuclein Expression). It also lowers levels of APP and all its fragments in APP/PS1 transgenic mice (A. F. Teich, et al., Alzheimer's & Dementia: Translational Research & Clinical Interventions 4 (2018) 37-45).

Buntanetap also lowers tau in vitro (Peter Davies Laboratory, Hofstra University, unpublished observation) and in vivo in Human tau mice (Peter Davies Laboratory, Hofstra University, unpublished observation). Buntanetap further lowers aSYN in vitro in neuroblastoma cells and in vivo in transgenic Parkinson's animals in the brain and in the gut (Kuo et. al. Am J Neurodegener Dis 2019; 8(1):1-15 www.AJND.us/ISSN:216 591X/AJND0086080: Translational inhibition of α-synuclein by Buntanetap (posiphen) normalizes distal colon motility in transgenic Parkinson mice).

Buntanetap's efficacy in treating neurodegeneration in animal models: Restored memory and learning in an APP/PS1 transgenic (tg) mouse model of AD; Restored memory and learning in a Ts65dn mouse model of Down syndrome (DS) [W. Mobley, UCSD, submitted 2020]; Preserved the retina in acute glaucoma [J Sundstrom; Hershey Medical School]; Restored colonic motility in a human SNCAA53T tg mouse model of PD (Kuo et. al., Am J Neurodegener Dis 2019; 8(1):1-15 www.AJND.us/ISSN:216 591X/AJND0086080: Translational inhibition of α-synuclein by Buntanetap normalizes distal colon motility in transgenic Parkinson mice); Preserved memory and learning in traumatic brain injury rats (M-F Chesselet, submitted 2020).

Buntanetap's reversal of the neurotoxic cascade: buntanetap's mechanism of action is related to APP, Tau, and αSYN expression being regulated by IRP1 and by iron and the way these proteins contribute to neurodegeneration by accumulating as toxic aggregates that impair axonal transport and synaptic transmission, causing inflammation, and, finally, leading to nerve cell death (as described previously). By reducing APP, Tau, and αSYN levels, buntanetap treatment prevented this toxic cascade. In support of this hypothesis, it has been shown that buntanetap: Normalized anterograde and retrograde vesicle transport in fully differentiated Down syndrome nerve cells [Chen et al]; Normalized impaired synaptic transmission in rat striatum after traumatic brain injury (TBI) (M-F Chesselet; UCLA) and hippocampus of APP/PS1 tg mice; lowered inflammation in human CSF of MCI subjects and in the rat brain after TBI; protected nerve cells in rat substantia Ingra after TBI and in a rat acute glaucoma model (J Sundstrom; Hershey Medical School); reduced the levels of Huntingtin Protein through Translational Suppression (Chen, X.-Q.; Barrero, C. A.; Vasquez-Del Carpio, R.; Reddy, E. P.; Fecchio, C.; Merali, S.; Deglincerti, A.; Fang, C.; Rogers, J.; Maccecchini, M. L. Posiphen Reduces the Levels of Huntingtin Protein Through Translation Suppression. Pharmaceutics 2021, 13, 2109. https://doi.org/10.3390/pharmaceutics13122109); and proved to be safe and promising in both Alzheimer's and Parkinson's Patients (Fang et al., Prev Alz Dis 2022)).

The AD field has been dominated by approaches to prevent APP processing or remove A3 in one of its many forms. These are downstream targets; buntanetap prevents the translational synthesis of the two main proteins involved in AD—APP and tau—and hence should remove all the downstream consequences produced by these proteins. Similarly, the PD field mostly focuses on inhibiting accumulation of αSYN aggregates and the effect of other proteins in this pathway, including LRRK or Parkin. Again, buntanetap prevents the synthesis of αSYN and thus it should stop the pathological cascade at the first step. Our data indicate that by normalizing the levels APP/Ap, Tau/phospho-Tau, and αSYN, buntanetap normalizes axonal transport, lowers inflammation, and protects nerve cells from dying. (Mobley 2020, submitted for publication; Chesselet 2020, submitted for publication).

### Example 2A

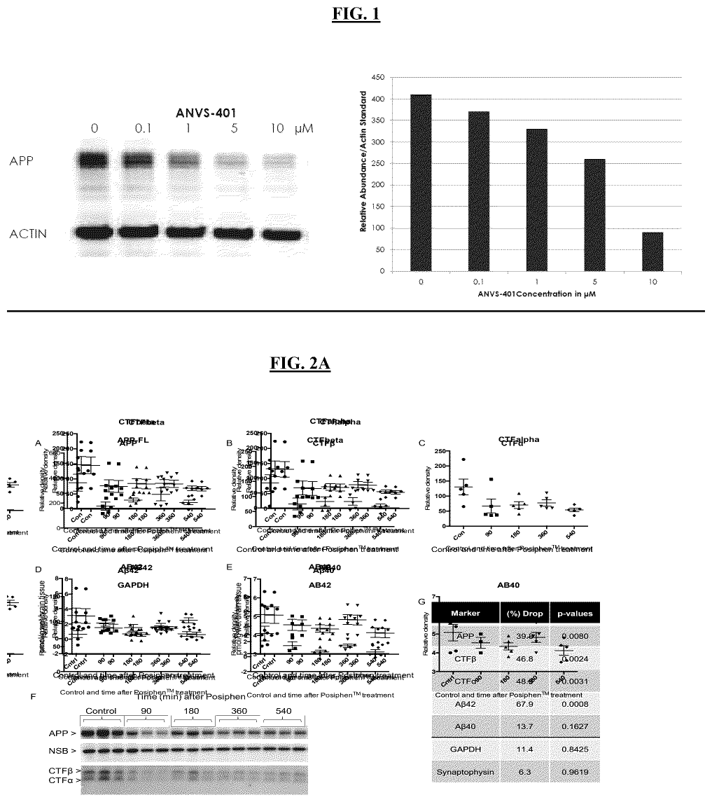

FIG. 1, APP in vitro. shows that buntanetap lowers APP in vitro in a dose-dependent manner in SH-SY-5Y human neuroblastoma cells. On the left is a Western blot showing buntanetap inhibition of APP in relation to Actin standard at concentrations of 0, 0.1, 1, 5 and 10 μM and a graph showing the same data plotted for statistical analysis purposes.

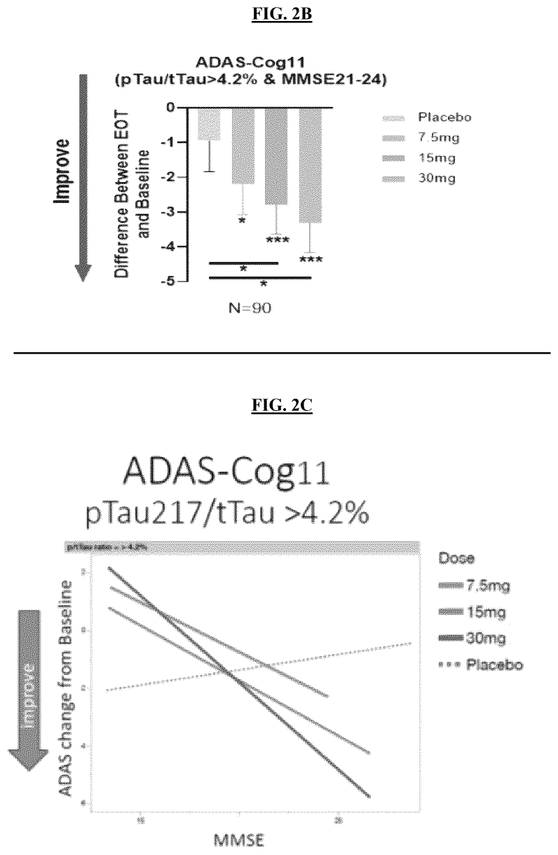

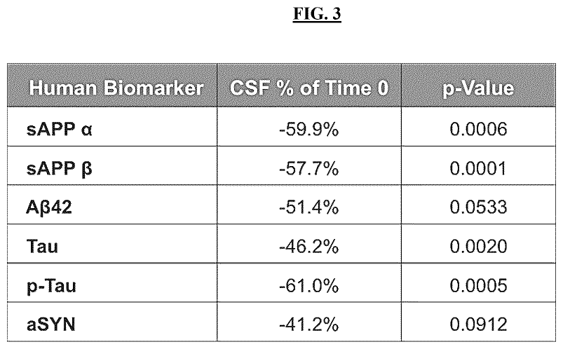

FIG. 2, APP in vivo. This study was conducted to demonstrate the effect of buntanetap in inhibiting the translation of APP and its fragments in an AD model in vivo. The Table in FIG. 2 shows that buntanetap treatment of APP/PS1 transgenic AD mice reduced APP and its fragments in hippocampus. GAPDH and Synaptophysin were loading controls. FIG. 2 also includes a collection of graphs showing relative density of APP plotted against control and time after buntanetap treatment; relative density of CTFβ plotted against control and time after buntanetap treatment; relative density of CTFα plotted against control and time after buntanetap treatment; A042 levels in brain tissue plotted against control and time after buntanetap treatment; and A040 levels in brain tissue plotted against control and time after buntanetap treatment. Finally, FIG. 2 also includes a Western blot showing levels of APP, NSB, CTFβ, and CTFα after buntanetap treatment over time (minutes). In APP/PS1 mice expressing human mutations associated with familial AD, the data show that buntanetap treatment reduced APP and all related peptides in hippocampus for at least 9 hours after the last dose.

### Example 2B

A randomized, double-blind, placebo-controlled Phase II/III study trial investigating the efficacy, safety, and tolerability of buntanetap in patients with mild to moderate AD was conducted. This was a dose-ranging study where patients received either one of three doses of buntanetap (7.5 mg, 15 mg, or 30 mg) or placebo on top of their standard of care for 12 weeks. In this study, over 700 patients were screened, a total of 353 patients were enrolled, and 325 patients completed the study across 54 sites in the US. The study included mild to moderate AD patients whose Mini Mental State Examination (MMSE) scores at baseline ranged from 14 to 24. (www.clinicaltrials.gov (NCT05686044)).

Beyond safety, the trial assessed the changes in two co-primary endpoints: Alzheimer's Disease Assessment Scale-Cognitive Subscale 11 (ADAS-Cog 11) and Alzheimer's Disease Cooperative Study Clinician's Global Impression of Change (ADCS-CGIC), which assess cognition and activities of daily living. The study monitored for safety and collected plasma to measure several biomarkers to assess the disease state, potential disease progression, and treatment effects.

A significantly higher improvement in ADAS-Cog 11 scores in each treatment dose relative to placebo for patients with mild AD was observed. The analysis focused on biomarker-positive early AD patients (MMSE 21-24, pTau217/tTau>4.2%) found that ADAS-Cog 11 was highly statistically significant at all 3 dose levels and in the combined dose levels compared to placebo as well as to baseline (FIG. 2B). The treatment response in the current study was not related to a patient's age or sex.

At the end of 3 months of treatment, placebo group demonstrated slight improvement (LSM(SE), 0.26 (0.91)), but not significantly different from baseline. However, all three buntanetap treatment groups showed statistically significant improvement from their corresponding baseline (7.5 mg improved 2.19 (0.87), p=0.013; 15 mg improved 2.79 (0.81), p=0.001; 30 mg improved 3.32 (0.82), P<0.001). Both 15 mg and 30 mg treatment groups also had a statistically significant improvement relative to placebo group (p=0.042 and 0.015 respectively). EOT—End of Treatment * P<0.05; ** P<0.01; ***P<0.001.

When the baseline MMSE scores for patients positive for AD according to their pTau217/tTau>4.2% ratio were subdivided, a dose-dependent relationship to MMSE at baseline was observed. It was concluded that the response to buntanetap treatment is more pronounced in mild AD patients than in those with more advanced AD. The response in the 30 mg dose treatment group R2=0.17 (R² or the coefficient of determination), p<0.001, indicates statistical significance of the MMSE score, which was not evident in the placebo group. FIG. 2C confirms the efficacy of buntanetap as previously shown in FIG. 2B.

It was concluded that was a three-fold difference in the proportion of participants who improved in the 30 mg group relative to placebo (Table 1A).

TABLE 1A
Responders vs Non-Responders

| | Responders | | Non-Responders | | |
|---|---|---|---|---|---|
| | (Number & | | (Number & | | Total |
| Dose | Percentage) | | Percentage) | | Number |
| Placebo | 6 | (27.27%) | 16 | (72.73%) | 22 |
| 7.5 mg Buntanetap | 14 | (73.68%)** | 5 | (26.32%) | 19 |
| 15 mg Buntanetap | 18 | (72.00%)** | 7 | (28.00%) | 25 |
| 30 mg Buntanetap | 21 | (87.50%)*** | 3 | (12.50%) | 24 |

**Contrast with Placebo, p value < 0.01,
***Contrast with Placebo, p value < 0.001

It was concluded that the data presented in FIGS. 2B and 2C demonstrates efficacy of buntanetap in early AD patients.

### Example 3

FIG. 3 is a study of subjects with mild cognitive impairment (MCI) for early proof of mechanism (POM) using a well-tolerated dose of buntanetap. Before and after 10 days of buntanetap administration to the MCI subjects, plasma, and cerebrospinal fluid (CSF) samples were obtained for analysis of levels of secreted (s) APPα and APPβ, and A042, Tau (total and phosphorylated), and inflammatory markers. FIG. 3 shows the reduction of APP/Ap, tau/phosphor-tau and alpha-synuclein in the spinal fluid of mildly cognitive impaired patients. In this study, buntanetap normalized these aggregating proteins in CSF of MCI subjects in accordance with the data seen in animals.

### Example 4

A randomized, double-blind, placebo-controlled Phase 2b trial investigated effects of liraglutide. (https://www.neurologylive.com/view/glp-1-agonist-liraglutide-shows-protective-effects-alzheimer-disease-phase-2-trial). The trial included 204 subjects with mild Alzheimer's disease. Half of the subjects received a daily subcutaneous injection of up to 1.8 mg liraglutide, and half of the subjects received placebo.

Cognitive testing was conducted before the start of treatment, at 24 weeks of treatment and at 52 weeks of treatment. Cognitive function was calculated as a composite score of 18 different tests of memory, comprehension, language, and spatial orientation.

It was concluded that for the subjects in the study who completed 52 weeks of treatment (treatment n=79, placebo n=87), those taking the drug saw a statistically significant slowing of cognitive decline (p<0.01).

Although the study was not powered to assess cognitive changes, it was concluded that subjects who received liraglutide had an 18% slower decline in cognitive function in a year compared to those who got the placebo.

It was further concluded that liraglutide appeared to reduce shrinking in the parts of the brain that control memory, learning, language, and decision-making by nearly 50% compared to placebo.

### Example 5

### Experiments with Combination of Buntanetap+Dulaglutide

### Material and Methods

Animals: Heterozygous double transgenic mice expressing both human APP (K670N:M671L) and human PS1 (M146L) (line 6.2) and their wild-type non-transgenic littermates, both males and females, were used for these experiments. They were obtained by a breeding colony maintained at the animal facility of Columbia University. They were genotyped through PCR on tail samples. Animals were injected i.p. from the age of 6 weeks for 1 month prior to starting behavioral assessment which lasted for 2 weeks during which treatment continued. Behavioral assessment examined spatial and associative memory. Finally, mice were sacrificed for electrophysiological assessment of synaptic function.

### Statistical Analysis

Mice were coded to blind investigators with respect to genotype and treatment. Results were expressed with Standard Error of the Mean (SEM). Level of significance was set for p<0.05. Results were analyzed by ANOVA analysis with post-hoc correction with the combination or genotype as main effect. Experiments were designed in a balanced fashion, and mice were trained and tested at each of the different conditions in 3 or 4 separate experiments.

### Electrophysiological Study

Mice were i.p. injected with the drug from the age of 8 weeks prior to plaque appearance until euthanasia for collecting slices at the age of 14-16 weeks. The treatment with Buntanetap was daily, whereas the treatment with dulaglutide was twice a week.

Electrophysiological analysis was performed on 400 μm hippocampal slices, cut with a tissue chopper and maintained in an interface chamber at 29° C. for 90 min prior to recording. Briefly, CA1 fEPSPs were recorded by placing both the stimulating and the recording electrodes in CA1 stratum radiatum. Basal synaptic transmission was assayed, either by plotting the stimulus voltages against slopes of fEPSP, or by plotting the peak amplitude of the fiber volley against the slope of the fEPSP. For LTP experiments, a 15 min baseline was recorded every min at an intensity that evokes a response ˜35% of the maximum evoked response. LTP was induced using θ-burst stimulation (4 pulses at 100 Hz, with the bursts repeated at 5 Hz and each tetanus including 3 ten-burst trains separated by 15 see). Data was subjected to statistical analyses to determine the effects of combination on inhibiting synaptic dysfunction.

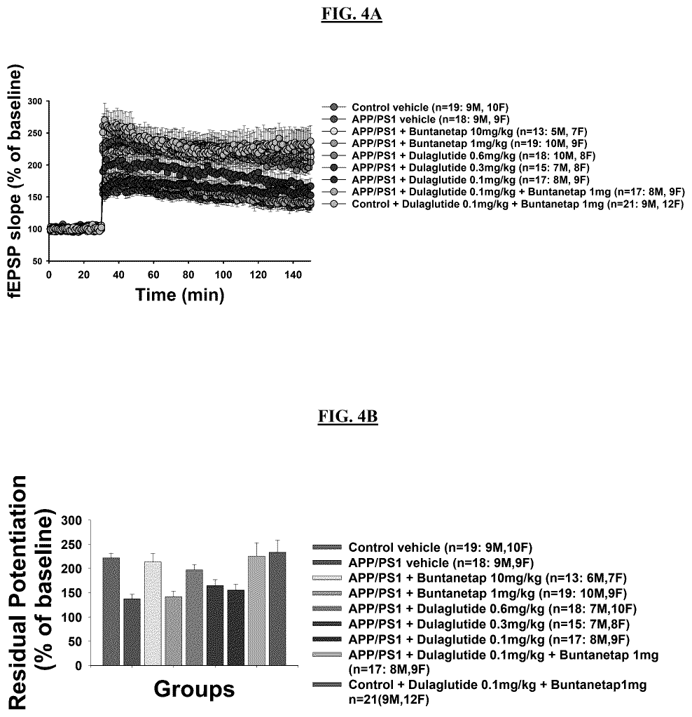

The results are graphically depicted in FIGS. 4A-4B.

Impairment of synaptic plasticity in hippocampal slices APP/PS1 transgenic mice measured through Long-term potentiation (LTP) was prevented by treatment with buntanetap (1 mg/Kg) plus dulaglutide (0.1 mg/Kg); whereas treatment with the same concentrations of buntanetap (1 mg/Kg) and dulaglutide (0.1 mg/Kg) alone did not ameliorate the synaptic defect in the transgenic animals. (1-way ANOVA followed by Bonferroni's comparisons: F_{(5,100)}=3.792, P=0.0035; Control non-transgenic+veh vs. APP/PS1+vehicle P=0.0052; APP/PS1+vehicle vs. APP/PS1+buntanetap (1 mg/Kg)+dulaglutide (0.1 mg/Kg) P=0.0045; APP/PS1 vs. APP/PS1+buntanetap (1 mg/Kg) P>0.9999; APP/PS1 vs. APP/PS1+dulaglutide (0.1 mg/Kg) P>0.9999; APP/PS1 vs. APP/PS1+buntanetap (10 mg/Kg) P=0.0327; APP/PS1 vs. APP/PS1+dulaglutide (0.6 mg/Kg) P=0.092; APP/PS1 vs. APP/PS1+dulaglutide (0.3 mg/Kg) P>0.9999). Data are means±s.e.m.).

It was concluded that the combination of translational inhibitor of neurotoxic aggregating proteins (TINAPs) Buntanetap with the GLP-1 agonist dulaglutide protects against amyloid-induced synaptic plasticity decline in vivo. (2-way ANOVA: Control nonTransgenic+veh vs. APP/PS1+vehicle F_{(1,35)}=41.88, P=0.0001; APP/PS1+vehicle vs. APP/PS1+buntanetap (1 mg/Kg)+dulaglutide (0.1 mg/Kg) F_{(1,33)}=12.77, P=0.0011; APP/PS1 vs. APP/PS1 Kg) F_{(1,34)}=20.65 P<0.0001; APP/PS1 vs. APP/PS1+dulaglutide (0.3 mg/Kg) F_{(1,31)}=5.507, P=0.0255).

The mean values of Residual Potential are provided in Table 3:

TABLE 3

| Treatment Group | Residual Potential |
|---|---|
| wt veh | 221.85 |
| APP/PS1 veh | 137.54 |
| APP/PS1 Buntanetap 10 mg/kg | 213.56 |
| APP/PS1 Buntanetap 1 mg/kg | 141.55 |
| APP/PS1 dulaglutide 0.6 mg/kg | 197.26 |
| APP/PS1 dulaglutide 0.3 mg/kg | 164.68 |
| APP/PS1 dulaglutide 0.1 mg/kg | 155.52 |
| APP/PS1 dulaglutide 0.1 mg/kg + | 225.43 |
| Buntanetap 1 mg/kg | |
| Wt dulaglutide 0.1 mg/kg + | 233.47 |
| Buntanetap 1 mg/kg | |
| (control) | |

The “wt veh” represents “normal”, and “APP/PS1 veh” represents “disease”. The difference between “normal” and “disease” is 84.31 (221.85-137.54=84.31), which represents 100% improvement.

The percent improvement in each treatment group, based on the change in the Residual Potential is provided in Table 4:

TABLE 4

| Treatment Group | % Improvement |
|---|---|
| APP/PS1 Buntanetap 10 mg/kg | 90.5 |
| APP/PS1 Buntanetap 1 mg/kg | 4.8 |
| APP/PS1 dulaglutide 0.6 mg/kg | 71.1 |
| APP/PS1 dulaglutide 0.3 mg/kg | 32.3 |
| APP/PS1 dulaglutide 0.1 mg/kg | 21.4 |
| APP/PS1 dulaglutide 0.1 mg/kg + | 104.6% |
| Buntanetap 1 mg/kg | |

It was concluded that the administration of the combination (buntanetap 1 mg/kg+dulaglutide 0.1 mg/kg) provided about 4.9-fold improvement over administration of dulaglutide alone (104.6/21.4=4.9) and about 21.8-fold improvement over administration of buntanetap alone (104.6/4.8=21.8).

It was also concluded that the therapeutic effect provided by the administration of the combination of buntanetap and dulaglutide, as compared to administration of each agent alone, was more than additive (104.6 vs. 26.2). The therapeutic effect provided by the administration of the combination was about 4 times greater than the additive affect (104.6/26.2=3.99).

### Behavioral Studies

Mice were i.p. injected with the drug from the age of 8 weeks prior to plaque appearance until behavioral assessment that started at 12 weeks of age and lasted for 2 weeks. The treatment with Buntanetap was daily, whereas the treatment with dulaglutide was twice a week (2-way ANOVA for repeated measures among all groups (day 2): F_{(8,118)}=4.716 P<0.0001).

Spatial memory was studied with the 2-day radial arm water maze (RAWM) test. The task is a hybrid of the Morris Water Maze (MWM) and the radial arm land maze. The motivation for the animals is immersion in water. The mouse needs to swim in 6 alleys (arms) radiating from a central area until it finds a hidden (submerged) platform at the end of one of the arms, based on visual cues placed in the room. In the old RAWM version of the task, the goal arm varies from day to day, requiring 21 days of training in wild-type mice to reach the learning criterion. In the new version of the task, the goal arm is kept constant for all trials, with a different start arm on successive trials, such that the learning criterion will be reached in 2 days. The first day of the protocol was a training day. Mice were trained to identify the platform location by alternating between a visible and a hidden platform in a goal arm. The final 3 trials on day 1 and all 15 trials on day 2 used a hidden escape platform to force mice to use spatial cues to identify the location of the goal arm. To avoid learning limitations imposed by exhausting practice and to avoid fatigue that may result from consecutive trials, spaced practice training was established by running the mice in cohorts of 4 and alternating different cohorts through the 15 training trials over 3 hours testing periods each day. On day 1, a visible platform was laced in a goal location. Mouse 1 of cohort 1 was gently placed in the pool near the perimeter of the wall of the first start arm (specified on a score sheet) and facing the center of the pool. The number of incorrect arm entries (entries in arms with no platform) was counted. If the animal enters the incorrect arm it was gently pulled back to the start arm. Each trial lasted up to 1 min. Failure to select an arm after 15 sec was counted as an error and the mouse was returned to the start arm. After 1 min, if the platform had not been located, the mouse was guided gently through the water by placing a hand behind it to direct it towards the platform. The mouse rested on the platform for 15 sec. After completing the trial, the mouse was removed from the pool, gently towel dried and placed back into its cage under a heat lamp. The goal platform location was different for each mouse. After all the mice in the first cohort had a trial to locate a visible platform, the platform was switched from visible to hidden. After each mouse from cohort 1 completed six alternating trials between visible and hidden platforms, the mice were left to rest under a heating source, and mice from the second cohort were tested in the same way. After completing the six alternating trials, mice from cohort 2 returned to their cages to rest. Next, mice from the first cohort completed trials 7-12 again using the alternating visible-hidden platform location. During resting time for mice from the first cohort, mice from the second cohort completed trials 7-12. At this point, all mice performed 3 hidden platform trials. On day 2, the same procedure was repeated as on day 1 for all 15 trials using only the hidden platform. For data analysis, averages for each mouse were calculated using blocks of 3 trials. Visible-platform training to test visual and motor deficits was performed in the same pool but without arms, with the platform marked with a black flag and positioned randomly from trial to trial. Each animal was allowed to swim for 1 min. Time to reach the platform and speed was recorded.

The combination effect on associative memory was assessed through fear conditioning (FC). For fear conditioning (FC) training, mice were placed in a conditioning chamber for 2 min before the onset of a tone (Conditioned Stimulus (CS), 30 sec, 85 dB sound at 2800 Hz). In the last 2 sec of the CS, mice were given a 2 sec, 0.7 mA mild foot shock (Unconditioned Stimulus, (US)) through the bars of the floor. After the US, the mice were left in the chamber for another 30 sec. Freezing behavior, defined as the absence of movements except for that needed for breathing, was scored using Noldus software. Contextual fear learning, a type of memory for which hippocampal function is indispensable, was evaluated 24 hrs after training by measuring freezing responses for 5 min in the same chamber where the mice have been trained. Cued fear learning, a type of memory that depends on amygdala function, was evaluated 24 hrs after contextual testing. The mice were placed in a novel context for 2 min (pre-CS test), after which they were given a CS for 3 min (CS test), and freezing behavior was measured during the first 30 sec that mimic the CS-US conditioning and the remaining 2.5 min. Sensory perception of the electric foot shock was examined in different groups of mice through the threshold assessment test. Briefly, the animals were placed in the conditioning chamber and the electric current (0.1 mA for 1 sec) was increased at 30 sec intervals from 0.1 mA to 0.7 mA. Threshold to flinching (first visible response to shock), jumping (first extreme motor response), and vocalized response was quantified for each animal by averaging the shock intensity at which each animal showed the behavioral response to that type of shock.

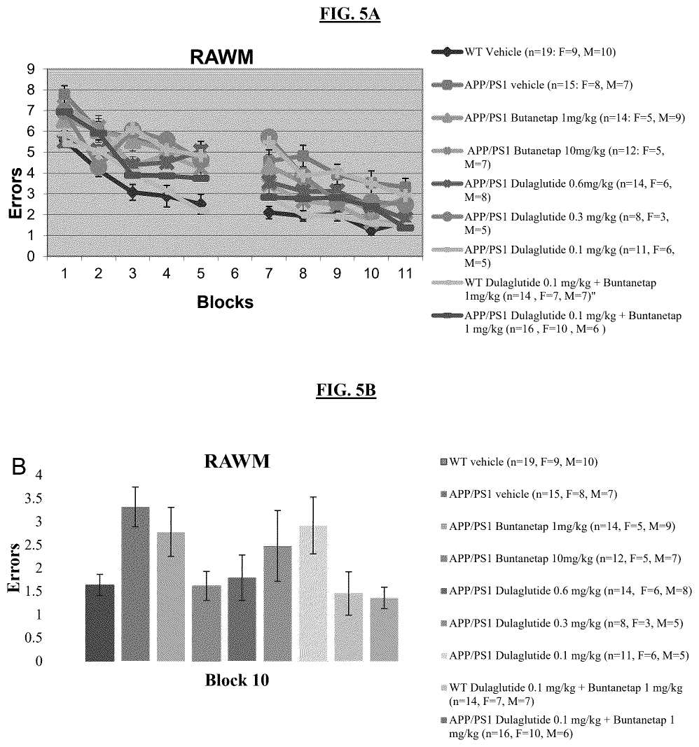

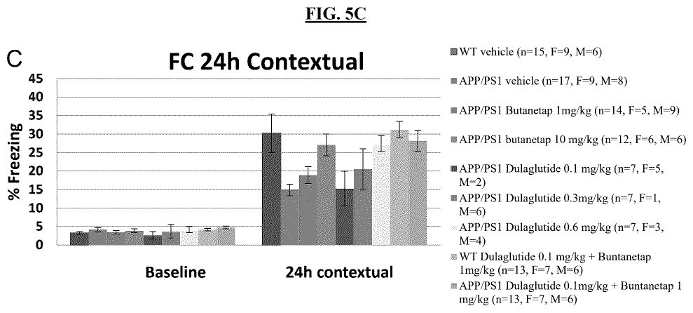

The results are graphically depicted in FIGS. 5A-5C.

The mean number of errors on the RAWM test are provided in Table 5:

TABLE 5

| Treatment Group | Meran Number of Errors |
|---|---|
| wt veh | 1.6 |
| APP/PS1 veh | 3.3 |
| APP/PS1 Buntanetap 10 mg/kg | 1.6 |
| APP/PS1 Buntanetap 1 mg/kg | 2.8 |
| APP/PS1 dulaglutide 0.6 mg/kg | 1.8 |
| APP/PS1 dulaglutide 0.3 mg/kg | 2.5 |
| APP/PS1 dulaglutide 0.1 mg/kg | 2.9 |
| APP/PS1 dulaglutide 0.1 mg/kg + | 1.5 |
| Buntanetap 1 mg/kg | |

The “wt veh” represents “normal”, and “APP/PS1 veh” represents “disease”. The difference between “normal” and “disease” is 1.7 (represents 100% improvement).

The percent improvement in each treatment group, based on the change in the Residual Potential is provided in Table 6:

TABLE 6

| Treatment Group | % Improvement |
|---|---|
| APP/PS1 Buntanetap 10 mg/kg | 100 |
| APP/PS1 Buntanetap 1 mg/kg | 29.4 |
| APP/PS1 dulaglutide 0.6 mg/kg | 88.2 |
| APP/PS1 dulaglutide 0.3 mg/kg | 47.1 |
| APP/PS1 dulaglutide 0.1 mg/kg | 23.5 |
| APP/PS1 dulaglutide 0.1 mg/kg + | 105.9 |
| Buntanetap 1 mg/kg | |

It was concluded that impairment of short-term spatial memory in APP/PS1 transgenic mice as measured through the 2-day radial arm water maze (RAWM) was prevented by treatment with Buntanetap (1 mg/Kg) plus dulaglutide (0.1 mg/Kg), whereas treatment with the same concentrations of Buntanetap (1 mg/Kg) and dulaglutide (0.1 mg/Kg) alone did not ameliorate the memory defect in the transgenic animals (FIG. 5A). FIG. 5B shows average number of errors during the last set of trials for the experiments shown in FIG. 5A (1-way ANOVA followed by Bonferroni's comparisons F_{(8,118)}=2.784 P=0.0074, Control nonTransgenic+veh vs. APP/PS1+vehicle P=0.0261; APP/PS1+vehicle vs. APP/PS1+buntanetap (1 mg/Kg)+dulaglutide (0.1 mg/Kg) P=0.0261; APP/PS1+vehicle vs. APP/PS1+buntanetap (10 mg/Kg) P=0.0591; APP/PS1+vehicle vs. APP/PS1+dulaglutide (0.6 mg/Kg) P=0.0888)

It was also concluded that impairment of associative memory in APP/PS1 transgenic mice measured through contextual fear conditioning (FC) was prevented by treatment with Buntanetap (1 mg/Kg) plus dulaglutide (0.1 mg/Kg); whereas treatment with the same concentrations of Buntanetap (1 mg/Kg) and dulaglutide (0.1 mg/Kg) alone did not ameliorate the memory defect in the transgenic animals (1-way ANOVA followed by Bonferroni's comparisons: F_{(8,96)}=3.932 P=0.0005, Control nonTransgenic+veh vs. APP/PS1+vehicle P=0.0020; APP/PS1+vehicle vs. APP/PS1+buntanetap (lmg/Kg)+dulaglutide (0.1 mg/Kg) P=0.0164; APP/PS1+vehicle vs. APP/PS1+buntanetap (10 mg/Kg) P=0.0448; APP/PS1+vehicle vs. APP/PS1+dulaglutide (0.6 mg/Kg) P=0.1300) (FIG. 5C).

It was concluded that the administration of the combination (buntanetap 1 mg/kg+dulaglutide 0.1 mg/kg) provided about 4.5-fold improvement in reduction of errors over administration of dulaglutide alone (105.9/23.5=4.5) and about 3.6-fold improvement in reduction of errors over administration of buntanetap alone (105.9/29.4=3.6).

It was also concluded that the effect provided by the administration of the combination of buntanetap and dulaglutide, as compared to administration of each agent alone, was more than additive (105.9 vs. 52.9). The therapeutic effect provided by the administration of the combination was about 2 times greater than the additive affect (105.9/52.9=2).

While the invention has been disclosed with reference to specific embodiments, it is apparent that other embodiments and variations of this invention may be devised by others skilled in the art without departing from the true spirit and scope of the invention. All patents and publications cited herein are incorporated by reference in their entirety. The appended claims are intended to be construed to include all such embodiments and equivalent variations.

What is claimed is:

1. A method for treating, inhibiting, preventing, slowing, or delaying the onset of a neurodegenerative disease comprising co-administering to a subject in need thereof:
(1) an amount of a compound selected from the group consisting of Formula (I), Formula (II) and Formula (III): 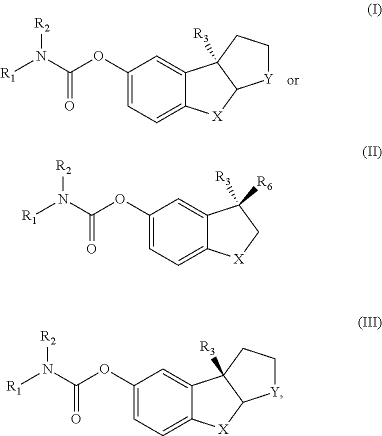
wherein,
in Formula (I) and Formula (II),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and
R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4;
wherein,
in Formula (III),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X is NR₅, wherein R₅ is C₂₋₈ alkenyl, C₂₋₈ alkynyl, or aralkyl;
Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl and wherein the compound having the Formula (I), Formula (II) or Formula (III) is the substantially pure (−)-enantiomer, the substantially pure (+)-enantiomer, or a racemic mixture of the (−)-enantiomer and (+)-enantiomers or a pharmaceutically acceptable salt thereof; and
(2) an amount of an antidiabetic agent.

2. The method of claim 1, wherein the compound is buntanetap or a pharmaceutically acceptable salt thereof.

3. The method of claim 1, wherein the compound and the antidiabetic agent are each independently administered orally, parenterally, intravenously, subcutaneously, sublingually, via suppository, nasally, topically, transdermally, or via implant under the skin.

4. The method of claim 1, wherein the compound is chronically administered, and the subject is experiencing type 1 diabetes or type 2 diabetes or type 3 diabetes or prediabetes.

5. The method of claim 4, wherein the subject is not demonstrating symptoms of a neurological disorder or a neurodegenerative disease.

6. The method of claim 2, wherein buntanetap or the pharmaceutically acceptable salt thereof is administered once-a-day orally in an amount from about 0.01 mg to about 2 mg.

7. The method of claim 6, wherein buntanetap or the pharmaceutically acceptable salt thereof is administered orally once-a-day in an amount from about 0.01 mg to about 0.8 mg.

8. The method of claim 6, wherein the administration provides a peak plasma circulating levels of buntanetap from about 1 ng/mL to about 40 ng/mL.

9. The method of claim 1, wherein the compound and the antidiabetic agent are administered separately but such that they provide overlapping therapeutic effects.

10. The method of claim 1, wherein the antidiabetic agent is selected from the group consisting of a glucagon-like peptide-1 receptor agonist, a GLP-1/glucagon dual agonist, a dual glucose-dependent insulinotropic polypeptide and GLP-1 receptor agonist, a sodium-glucose cotransporter 2 inhibitor, a dipeptidyl peptidase-4 inhibitor, a biguanide, a sulfonylurea, sodium glucose cotransporter 2 inhibitor, alpha-glucosidase inhibitor, dopamine-2 agonist, a meglitinide, a thiazolidinedione, an amylinomimetic, a type-3 antidiabetic agent, and combinations thereof.

11. The method of claim 1, wherein the subject has type-2 diabetes or prediabetes.

12. The method of claim 2, wherein the antidiabetic agent is a GLP-1 agonist.

13. The method of claim 12, wherein the GLP-1 agonist is dulaglutide or a pharmaceutically acceptable salt thereof.

14. The method of claim 1, wherein the compound is administered in an amount from about 0.1 mg to about 60 mg, and the antidiabetic agent is administered in an amount from about 0.01 μg to about 3000 mg.

15. The method of claim 14, wherein the compound is buntanetap or a pharmaceutically acceptable salt thereof.

16. A pharmaceutical composition, comprising
an amount of a compound selected from the group consisting of Formula (I), Formula (II) and Formula (III): 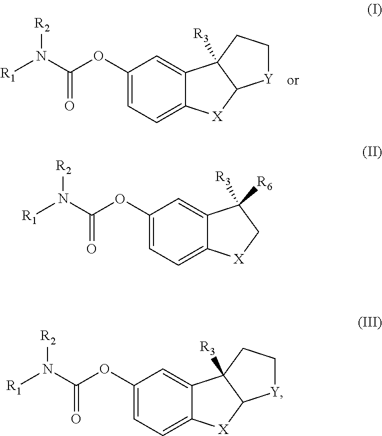
wherein,
in Formula (I) and Formula (II),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and
R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4;
wherein,
in Formula (III),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X is NR₅, wherein R₅ is C₂₋₈ alkenyl, C₂₋₈ alkynyl, or aralkyl;
Y is selected from C(H)R₄ or NR₅, wherein R₄ and R₅ are, independently, hydrogen, branched or straight chain C₁₋₈alkyl or heteroalkyl, alkenyl, or C₂-C₅ alkynyl, aralkyl and wherein the compound having the Formula (I), Formula (II) or Formula (III) is the substantially pure (−)-enantiomer, the substantially pure (+)-enantiomer, or a racemic mixture of the (−)-enantiomer and (+)-enantiomers or a pharmaceutically acceptable salt thereof; and
an amount of an antidiabetic agent.

17. The pharmaceutical composition of claim 14, wherein the compound is buntanetap or a pharmaceutically acceptable salt thereof.

18. The pharmaceutical composition of claim 16, wherein the antidiabetic agent is a glucagon-like peptide-1 receptor agonist.

19. An oral dosage form comprising the pharmaceutical composition of claim 16 and at least one pharmaceutically acceptable excipient.

20. The method of claim 1, wherein the neurodegenerative disease is selected from a group consisting of Alzheimer's disease, a tauopathy, chronic traumatic encephalopathy, frontotemporal dementia, Parkinson's disease, an alpha-synucleopathy, Prion's disease, Down Syndrome, Huntington's disease, multiple sclerosis, and Amyloid Lateral Sclerosis.
